(19) United States
(12) Patent Application Publication
(10) Pub. No.: US 2026/0144897 A1
(43) Pub. Date: May 28, 2026

(54) Methods of Targeting Mutant Cells

(71) Applicant: President and Fellows of Harvard College, Cambridge, MA (US)

(72) Inventors: Markus Basan, Brookline, MA (US); Avik Mukherjee, Brookline, MA (US)

(21) Appl. No.: 18/707,262

(22) PCT Filed: Nov. 10, 2022
(86) PCT No.: PCT/US2022/079646

§ 371 Date: May 3, 2024

Related U.S. Application Data
(60) Provisional application No. 63/278,200, filed on Nov. 11, 2021.

(51) Int. Cl. A61K 48/00; C12N 9/22; C12N 15/11; C12N 15/86

(52) U.S. Cl. CPC A61K 48/0058; C12N 9/226; C12N 15/11; C12N 15/86; C12N 2310/20; C12N 2710/10043

(57) ABSTRACT

The present disclosure provides methods and systems for targeting cell with mutations.

### STATEMENT OF GOVERNMENT INTERESTS

This invention was made with government support under GM137895 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 8, 2022, is named “Sequence_Listing_010498_01500_ST26” and is 75 KB in size.

### FIELD

The present invention relates in general to methods of targeting cells including a mutation for treatment of such cells. The present invention also relates to methods of discriminating between cells including a mutation and cells which lack the mutation for purposes of expressing a treatment molecule.

### BACKGROUND

Cancer cells emerge from healthy cells in the body by genetic mutations that can be identified through DNA sequencing technology. Methods of targeting cells with a mutation are described in Cheung et al., Laboratory Investigation (2018) 98:968-976 where a single nucleotide mutation creates a PAM that can be targeted by a CRISPR/Cas9 system for cleavage.

There is a need for specifically targeting cells, such as cancer cells, for expression of a treatment molecule and without cells that lack the mutation, i.e. healthy cells, also expressing the treatment molecule.

### SUMMARY

According to one aspect, the present disclosure provides methods and systems for targeting cells for expression of a treatment molecule that have a mutation in cellular DNA. Exemplary mutations include without limitation a point mutation, a frameshift mutation, a translocation, an inversion, an insertion, a deletion, a duplication, a nucleic acid encoding a protein fusion or a viral integration and the like. Such mutations are targetable by the nucleic acid constructs described herein for insertion into cellular DNA and expression of a treatment molecule. The construct is designed such that expression of the treatment molecule by cells which lack such a mutation or mutations in inhibited. According to one aspect, cells which include a targeted mutation or mutations express the treatment molecule while cells which lack the targeted mutation or mutation do not express the treatment molecule.

According to one aspect, such mutant cells are directly treated, i.e. express the treatment molecule, while cells which lack the mutation, i.e. off-target cells, are not directly treated, i.e. do not express the treatment molecule or at least do not significantly express the treatment molecule, such that the method discriminates treatment between cells which have the target mutation and cells which lack the target mutation. Methods and systems described herein allow for the selective treatment of mutant cells compared to cells which lack the mutation. A construct encoding a treatment molecule, such as a toxin, is delivered to the mutant cells where the construct is then inserted into cellular DNA and expressed in the mutant cells to provide the treatment molecule. According to one aspect, the construct is designed such that expression of the construct is not capable or is inhibited until integrated into the genome of the cell. According to one aspect, the construct is designed such that expression of the construct is not capable or is inhibited in cells which lack the mutation. Accordingly, methods and systems described herein target mutant cells for treatment and discriminate between cells which have a mutation and cells which lack the mutation. Mutations are used to selectively deliver and express constructs encoding treatment molecules to such mutant cells while such constructs are inhibited or are not expressed in off-target cells lacking such mutations. In this way, mutant cells can be highly specifically targeted for treatment, without significantly harming cells with lack the mutation, such as healthy cells which may be adjacent to the mutant cells. According to one aspect, the construct is designed to prevent expression of the treatment molecule by a cell which lacks the mutation. Expression of the treatment molecule is dependent on the presence of the mutation in a target cell.

Further features and advantages of certain embodiments of the present disclosure will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is a schematic diagram depicting a general method of targeting cells with a mutation in cellular DNA.

FIG. 2A and FIG. 2B are directed to mutation-specific editing and protein expression achieved by an exemplary nucleic acid construct. Green by expression of GFP indicates the presence of the construct and the expression of Cas9 in those cells. The nucleic acid construct includes a first nucleic acid encoding mcherry, referred to herein as “a cargo protein” that is delivered to a cell with mutation specificity (red) but should not be expressed in cells lacking this specific mutation. FIG. 2A Left: Roughly 50% of H1975 cells (cell line with the targeted specific mutation) that were successfully transfected with the nucleic acid construct, as indicated by GFP expression, started to express mcherry, indicating successful editing, and integration in the chromosome, leading to expression of the cargo protein. FIG. 2B Right top: Roughly half of the H1975 cells that were successfully transfected with the nucleic acid construct, as indicated by GFP expression, did not express mcherry indicating that these cells were not successfully edited. FIG. 2B Right bottom: While H1650 cells (cell line lacking the specific mutation) were successfully transfected and Cas9 was expressed, as indicated by expression of GFP, no expression of the cargo protein mcherry was observed in any cell. This means that there was no detectable leaky expression of the cargo protein from the construct nor off-target editing of cells lacking the specific mutation.

FIG. 3A, FIG. 3B and FIG. 3C are directed to mutation-specific virus replication enabled by mutation-specific gene editing and mutation-specific expression of viral proteins for replication. Ad5 Iva2 virus was engineered for mutation-specific replication in cells carrying a single oncogenic point mutation in the epidermal growth factor receptor (“EGFR”) gene. Iva2 is required for viral replication. The native Iva2 gene was deleted from the viral genome and instead the Iva2 gene was used as the cargo protein to be integrated and expressed in a mutation-specific manner after gene editing using the construct of FIG. 2A, which was included in the viral genome. GFP expression in-frame with Cas9 could be used to detect the presence of viral DNA and track the propagation of the viral infection in cell culture. FIG. 3A and FIG. 3B: two weeks after transfection, single transfected H1975 cells (cell line with the targeted specific-mutation) matured into clusters of GFP-expressing fluorescent cells, indicating successful viral replication based on gene editing. This cluster contains about 70 cells after two weeks. There were many other clusters in the cell culture and the infection continued to propagate for about 12 weeks, at which point the experiment was discontinued and the virus was harvested. FIG. 3C: In the H1650 control cells that carry wild type EGFR (lacking the specific mutation), no growing fluorescent clusters indicative of viral replication were observed. This is expected, because without the correct mutation in these cells, the Iva2 protein essential for viral replication should not be expressed and the infection is unable to propagate.

FIG. 4 depicts an exemplary mutation-specific gene delivery and expression construct.

FIG. 5 depicts an exemplary mutation-specific gene editing and viral replication construct.

FIG. 6 depicts a map of an exemplary plasmid useful in the methods described herein.

FIG. 7 depicts various mutations which can be targeted by the methods of the present disclosure. (SEQ ID NO:1-7)

FIG. 8 depicts various mutations which can be targeted by the methods of the present disclosure. (SEQ ID NO:8-13)

FIG. 9 is a schematic diagram of a core construct including a cargo nucleic acid sequence to be delivered through an adenoviral genome.

FIG. 10 depict images of the propagation of mutation-specific replicating adenovirus in NCI H-1975 cells including a target mutation.

FIG. 11 depict images of the lack of propagation of mutation-specific replicating adenovirus in NCI H-1650 cells that lack a target mutation.

FIG. 12 is a graph quantifying the infected cells expressing Cas9 GFP from FIG. 10 and FIG. 11.

FIG. 13 depict images of example clusters in NCI H-1975 cells.

FIG. 14 is a graph quantifying the total number of observed viral clusters in NCI H-1650 and NCI H-1975 cells about 1.5 months after the beginning of the first of two separate experiments described in Example III.

### DETAILED DESCRIPTION

As used in this specification and the appended claims, the singular forms “a”, “an” and “the” include plural referents unless the context clearly indicates otherwise. Thus, for example, reference to “a treatment molecule” includes more than one treatment molecule, and reference to “an expression element” includes more than one expression element.

It is further to be understood that use of “or” means “and/or” unless stated otherwise. Similarly, “comprise,” “comprises,” “comprising” “include,” “includes,” and “including” are interchangeable and not intended to be limiting. Also, where descriptions of various embodiments use the term “comprising,” those skilled in the art would understand that in some specific instances, an embodiment can be alternatively described using language “consisting essentially of” or “consisting of.”

The foregoing general description, including the drawings, and the following detailed description are exemplary and explanatory only and are not restrictive of this disclosure. The section headings used herein are for organizational purposes only and not to be construed as limiting the subject matter described.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings:

“Gene” as used herein refers to a nucleic acid region, also referred to as a transcribed region, which expresses a polynucleotide, such as an RNA. The transcribed polynucleotide can have a sequence encoding a polypeptide, such as a functional protein, which can be translated into the encoded polypeptide when placed under the control of an appropriate regulatory region. A gene may comprise several operably linked fragments, such as a promoter, a 5′ leader sequence, a coding sequence and a 3′ nontranslated sequence, such as a polyadenylation site. A chimeric or recombinant gene is a gene not normally found in nature, such as a gene in which, for example, the promoter is not associated in nature with part or all of the transcribed DNA region. “Expression of a gene” refers to the process wherein a gene is transcribed into an RNA and/or translated into a functional protein.

“Gene delivery” or “gene transfer” refers to methods for introduction of recombinant or foreign DNA into host cells. The transferred DNA can remain non-integrated or preferably integrates into the genome of the host cell. Gene delivery can take place for example by transduction, using viral vectors, or by transformation of cells, using known methods, such as electroporation, cell bombardment.

“Transgene” refers to a gene that has been introduced into a host cell. The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell.

“Transduction” refers to the delivery of a nucleic acid molecule into a recipient host cell, such as by a gene delivery vector, such as rAAV. For example, transduction of a target cell by a rAAV virion leads to transfer of the rAAV vector contained in that virion into the transduced cell. “Host cell” or “target cell” refers to the cell into which the nucleic acid delivery takes place.

“Functional protein” includes variants, mutations, homologues, and functional fragments of the full length proteins. One of skill will readily be able to construct proteins homologous to the full length proteins which retain the activity, in whole or in part, of the full length protein.

“Vector” refers generally to nucleic acid constructs suitable for cloning and expression of nucleotide sequences. One example of a vector is a viral vector. The term vector may also sometimes refer to transport vehicles comprising the vector, such as viruses or virions, which are able to transfer the vector into and between host cells.

“AAV vector” or “rAAV vector” refers to a recombinant vector derived from an adeno-associated virus serotype, such as AAV1, AAV2, AAV3, AAV4, AAV5, AAV6, AAV7, AAV8, AAV9, AAV10, AAV11, AAV12, AAV2.5, AAvDJ, AAVrh10.XX and others. rAAV vectors can have one or preferably all wild type AAV genes deleted, but still comprise functional ITR nucleic acid sequences. Functional ITR sequences are necessary for the replication, rescue and packaging of AAV virions. The ITR sequences may be wild type sequences or substantially identical sequences (as defined below) or may be altered by for example in insertion, mutation, deletion or substitution of nucleotides, as long as they remain functional.

“Therapeutically effective amount” refers to an amount effective, at dosages and for periods of time necessary, to achieve the desired therapeutic result, which may be cell death. A therapeutically effective amount of a parvoviral virion or pharmaceutical composition may vary according to factors such as the disease state, age, sex, and weight of the individual, and the ability of the parvoviral virion or pharmaceutical composition to elicit a desired response in the individual. Dosage regimens may be adjusted to provide the optimum therapeutic response, such as death of deleterious cells as described herein.

“Nucleic acid” includes any molecule composed of or comprising monomeric nucleotides. The term “nucleotide sequence” may be used interchangeably with “nucleic acid” herein. A nucleic acid may be an oligonucleotide or a polynucleotide. A nucleic acid may be a DNA or an RNA. A nucleic acid may be a gene. A nucleic acid may be chemically modified or artificial. Artificial nucleic acids include peptide nucleic acid (PNA), Morpholino and locked nucleic acid (LNA), as well as glycol nucleic acid (GNA) and threose nucleic acid (TNA). Each of these is distinguished from naturally-occurring DNA or RNA by changes to the backbone of the molecule. Also, phosphorothioate nucleotides may be used.

“Nucleic acid construct” is herein understood to mean a man-made nucleic acid molecule resulting from the use of recombinant DNA technology. A nucleic acid construct is a nucleic acid molecule, either single- or double-stranded, which has been modified to contain segments of nucleic acids, which are combined and juxtaposed in a manner, which would not otherwise exist in nature. A nucleic acid construct may be within a “vector”, i.e. a nucleic acid molecule which is used to deliver exogenously created DNA into a host cell. A nucleic acid construct may also broadly refer to the vector including the nucleic acid construct. One type of nucleic acid construct is an “expression cassette” or “expression vector”. These terms refers to nucleotide sequences that are capable of effecting expression of a gene in host cells or host organisms compatible with such sequences. Expression cassettes or expression vectors typically include at least suitable transcription regulatory sequences and optionally, 3′ transcription termination signals. Additional factors necessary or helpful in effecting expression may also be present, such as expression enhancer elements. A nucleic acid construct can also be a vector in which it directs expression or repression of a protein by operating as RNA instead of DNA. In the case of increasing expression of a target protein this nucleic acid construct can be mRNA or similar in which the cell or more specifically the ribosome would recognize and create many copies of the protein. In the case of repressing expression of a target sequence the RNA can be in the form that acts through preventing the ribosome from creating protein, this can be done through mechanisms of RNAi or shRNA or miRNA or Pri-miRNA.

“Operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

“Naturally occurring sequence” or “native sequence” as used herein refers to a polynucleotide or amino acid isolated from a naturally occurring source. Included within “native sequence” are recombinant forms of a native polypeptide or polynucleotide which have a sequence identical to the native form.

“Mutant” or “variant” as used herein refers to an amino acid or polynucleotide sequence which has been altered by substitution, insertion, and/or deletion. In some embodiments, a mutant or variant sequence can have increased, decreased, or substantially similar activities or properties in comparison to the parental sequence.

Aspects of the present disclosure are directed to methods and systems that are capable of targeting cells on the basis of a mutation and, accordingly, discriminate between target cells having a mutation in cellular DNA and off-target cells which lack the mutation in cellular DNA. According to one aspect, a nucleic acid construct is designed that includes a first nucleic acid encoding a treatment molecule that is capable of significant expression of the first nucleic acid encoding the treatment molecule in cells which have the mutation and is incapable of expression in cells which lack the mutation, or at the very least is capable of insignificant expression of the first nucleic acid encoding the treatment molecule. The design of the nucleic acid construct allows high specificity in targeting cells based on specific mutations in those cells, resulting in no or minimal off-target expression in cells lacking the mutation. In this manner, the nucleic acid construct is integrated into the genome of a cell having the target mutation or mutations, but is not integrated into the genome of a cell lacking the target mutation or mutations, or at least is not significantly integrated into the genome of a cell lacking the target mutation or mutations. According to one aspect, the nucleic acid construct, the first nucleic acid and/or the delivery vector lack one or more expression elements such that expression of the first nucleic acid is inhibited before integration into cellular DNA, such as genomic DNA. According to one aspect, the nucleic acid construct, the first nucleic acid and/or the delivery vector include one or more expression elements but also include one or more expression inhibition elements to inhibit expression of the first nucleic acid encoding the treatment molecule such that expression of the first nucleic acid is inhibited before integration into cellular DNA, such as genomic DNA. After integration, the one or more expression inhibition elements can be removed to allow expression of the first nucleic acid encoding the treatment molecule. On this basis, methods described herein allow discrimination between expression in mutant cells and cells which lack the mutation.

According to one aspect, the present disclosure is directed to a method of treating a collection of cells including a target mutated cell or cells including a first target mutation in cellular DNA and a plurality of cells lacking the first target mutation. The first target mutation can be a plurality of mutations as described further herein and need not be a point mutation. The method includes administering to the collection of cells (1) a nucleic acid construct having (i) a first nucleic acid encoding a treatment molecule wherein the nucleic acid construct optionally lacks one or more expression elements, such as for stable expression of the first nucleic acid before integration of the construct and optionally targets the first target mutation. Alternatively, the nucleic acid construct includes one or more expression constructs for expression of the first nucleic acid but also includes one or more expression inhibition constructs that inhibit expression of the first nucleic acid. According to one aspect, the one or more expression inhibition constructs can be removed thereby allowing expression of the nucleic acid encoding the treatment molecule. The nucleic acid construct optionally includes a second nucleic acid encoding a gene editor, wherein, if present, the gene editor is expressed, and wherein the gene editor optionally targets the first target mutation and cleaves the cellular DNA at a target integration site. It is to be understood that a gene editor is not necessary to carry out the methods described herein, as it is well known that nucleic acid constructs can be inserted into cellular DNA without cutting or nicking the cellular DNA. It is well known that nucleic acid constructs can be spontaneously inserted into cellular DNA. See for example, Capecchi MR. Altering the genome by homologous recombination. [Review] Science. 1989; 244:1288-92; Thomas K R, Capecchi M R. Site-directed mutagenesis by gene targeting in mouse embryo-derived stem cells. Cell. 1987; 51:503-12; Doetschman T, Gregg R G, Maeda N, Hooper M L, Melton D W, Thompson S, Smithies O. Targeted correction of a mutant HPRT gene in mouse embryonic stem cells. Nature. 1987; 330:576-8; Capecchi M R. High efficiency transformation by direct microinjection of DNA into cultured mammalian cells. Cell. 1980; 22:479-88; Folger K R, Wong E A, Wahl G, Capecchi M R. Patterns of integration of DNA microinjected into cultured mammalian cells: evidence for homologous recombination between injected plasmid DNA molecules. Mol Cell Biol. 1982; 2:1372-87.

According to the present disclosure, the nucleic acid construct and/or the gene editor targets the first target mutation. The first nucleic acid encoding the treatment molecule is integrated into the target integration site of the cellular DNA of the target mutated cell. The first nucleic acid encoding the treatment molecule is expressed after integration. The treatment molecule treats the target mutated cell.

According to one aspect, the present disclosure contemplates mutation-specific expression of protein toxin molecules in cancer cells.

According to one aspect, the present disclosure contemplates mutation-specific replication of oncolytic viruses by expression of viral proteins in a mutation-specific manner.

According to one aspect, the present disclosure contemplates targeted modification of signal transduction pathways for therapeutics in a mutation-specific manner

According to one aspect, the present disclosure contemplates mutation-specific killing of specific immune cells (B cell or T cells), causing autoimmune disorders or allergies, by targeting their V(D)J recombination sequence.

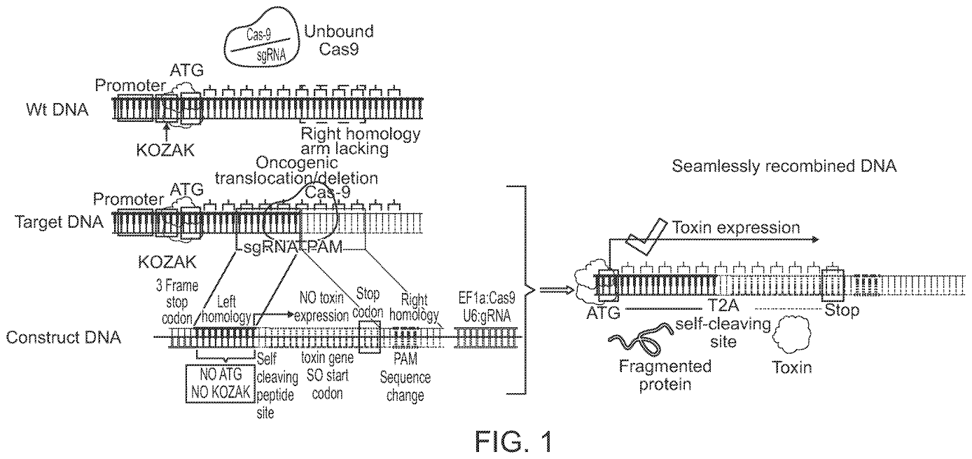

Methods are provided according to each of the above aspects. According to one exemplary embodiment according to the present disclosure and as depicted in schematic in FIG. 1, the DNA sequence of a cargo gene (e.g. encoding a toxin) without a promoter, start codon or Kozak sequence, preceded by stop codons in all three frames and a left homology region in which all start codons have been modified to prevent any leaky protein expression from the delivery construct is delivered to mutation-specific target cells. The cargo gene is preceded by a DNA sequence encoding a self-cleaving peptide and followed by a right homology region. A gene editor, such as a CRISPR-Cas system with guide RNA is included with the construct. To target ubiquitously occurring oncogenic gene fusions, translocations, or deletion mutations, the guide RNA-PAM sequence can be chosen to overlap the chromosome break, preventing any cutting in wildtype cells due to absence of this sequence. The cargo gene is then seamlessly integrated into the target DNA, downstream of a promoter and in-frame with a start codon. The cargo protein will now be expressed. Not only will no DNA-cutting take place in wildtype cells, due to the missing guide RNA-PAM sequence, but there are no matching homology arms, restricting off-target integration and expression. Point mutations can be targeted with the same strategy if the mutation creates a PAM site for one of the available Cas enzymes. Frameshift mutations can be targeted even when they do not create a PAM site by placing the cargo gene in-frame with the frameshift.

### I. Target Cells

According to one aspect, the collection of cells can be homogenous, i.e. of the same cell type. According to one aspect, the collection of cells can be heterogeneous, i.e. of different cell type. It is to be understood that target cells include a mutation or unique DNA sequence that can be targeted by the nucleic acid construct or constructs, as described herein. It is to be understood that the target mutated cells include a mutation that can be targeted and that can be the basis for discriminating between target mutated cells and off-target cells, i.e. wild type cells or cells which do not have the mutation. According to one aspect, one or more or a plurality of mutations in a target mutated cell can be targeted. In this manner, several constructs targeting several mutations can be introduced into the cell for expression of the same or different treatment molecules.

According to one aspect, the present disclosure contemplates any cell having one or mutations or a plurality of mutations as described herein that can be targeted by a nucleic acid construct as described herein. Deleterious cells according to the present disclosure include any cell into which foreign nucleic acids can be introduced and expressed as described herein. It is to be understood that the basic concepts of the present disclosure described herein are not limited by cell type. In some embodiments, the cell is a eukaryotic cell or prokaryotic cell. In some embodiments, the eukaryotic cell is a plant, yeast, mammalian, stem cell, human cell, or human stem cell. Exemplary cells include a neoplastic cell, a cancer cell, an immune cell, a virus-infected cell, a pathogen-infected cell, and the like. Exemplary target mutated cells include a bacterial cell, an insect cell, a plant cell or an animal cell, including a mammalian cell, such as a human cell.

### II. Cellular DNA

According to one aspect, the present disclosure contemplates targeting cellular DNA including a mutation. Exemplary cellular DNA includes genomic DNA, mitochondrial DNA, plasmid DNA, exogenous DNA, foreign DNA or viral DNA. According to one aspect, the cellular DNA is a gene.

### III. Treatment Molecules

According to one aspect, a treatment molecule can include any protein or RNA used to treat a mutated cell as desired. The treatment molecule is encoded by a nucleic acid sequence which is then expressed by a cell with a target mutation or mutations. According to one aspect, expression of the nucleic acid sequence can occur upon integration into the cellular DNA using the cellular DNA expression elements such that the treatment molecule is produced by the cell. According to one aspect, expression can be induced after integration into the cellular DNA, such as by an inducible gene expression system or by removing an expression inhibitor from the nucleic acid construct as described herein.

According to one aspect, a treatment molecule includes a protein toxin. Exemplary protein toxins known to those of skill in the art include ricin, Shiga toxin (Stx) 1, Shiga toxin (Stx) 2, Cholera toxin, Melittin, Phospholipases A and C, Streptolysin O and S, Pertussigen, Clostridium difficile TcdB, Sphingomyelinase C, Staphylococcus aureus alpha toxin, Staphylococcus aureus beta toxin, Staphylococcus aureus delta toxin and the like. It is to be understood that the above toxins are exemplary and non-exhaustive and that other toxins known to those of skill in the art can be selected for a specific application, depending for example on the level of toxicity required and tolerated, as well as the mechanism of action of the toxin.

Exemplary methods described herein include targeted killing or growth inhibition of deleterious cells, such as cancer cells (including neighboring deleterious or cancer cells) by expression of protein toxins or combinations of toxins in a mutation-specific manner, i.e. in deleterious cells having one or more or a plurality of mutations. Exemplary methods include targeted killing or inhibition of specific cancer cells having one or more or a plurality of mutations or immune cell populations for treating immune disorders based on unique DNA sequences from immune cell diversity in these populations.

According to one aspect, edited deleterious cells, even a small number of edited deleterious cells, can be used to produce high levels of protein toxins that can kill neighboring non-edited deleterious cells. Bacterial AB toxins (e.g. ricin, Shiga toxin) are exemplary, because they are activated only when processed after endocytosis, allowing high levels of toxin production by edited cells before they succumb to the toxin.

According to one aspect, a treatment molecule includes an immune antigen such as bacterial LPS, peptidoglycan, and flagellar protein or a component thereof which can be ectopically expressed or an immunomodulatory factor such as a cytokine or a component thereof. Immune antigen and immune modulatory factors include those that can evoke heightened immune response and cause infiltration of dendritic cells, macrophages, neutrophils, and NK cells. See for example Huang X, Pan J, Xu F, et al. Bacteria-Based Cancer Immunotherapy. Adv Sci (Weinh). 2021; 8 (7): 2003572. Published 2021 Feb. 10. doi: 10.1002/advs.202003572. It is to be understood that the above treatment molecules are exemplary and non-exhaustive and that other treatment molecules known to those of skill in the art can be selected for a specific application. Aspects of the present disclosure include targeted killing or growth inhibition of deleterious cells including one or more or a plurality of mutations, such as cancer cells, by expression of immune antigens, immunomodulatory factors other therapeutics in, for example, cancer cells in a mutation-specific manner.

According to one aspect, a treatment molecule is an RNA, an immune antigen, an immunomodulatory factor, a nanobody, a modulator of a signal transduction pathway, an enzyme or enzyme subunit, a dominant negative form of a cell signaling pathway intermediate, and a transcription factor, and the like.

### IV. Target Mutations

According to one aspect, the present disclosure contemplates any mutation or mutations or plurality of mutations or unique DNA sequence within a cell that can be targeted.

Exemplary mutations include a point mutation, a frameshift mutation, a translocation, an inversion, an insertion, a deletion, a duplication, a chromosomal translocation or a nucleic acid encoding a protein fusion and the like. Such mutations are known to those of skill in the art or can be determined by routine literature search or can be found for specific cells using genome sequencing technology. See for example, Edwards P A. Fusion genes and chromosome translocations in the common epithelial cancers. J Pathol. 2010 January; 220 (2): 244-54. doi: 10.1002/path.2632. PMID: 19921709.

CRISPR-Cas9 mediated genome editing enables induction of a double stranded DNA break at a specific sequence, provided a PAM sequence (5′NGA3′ or 5′ NGG3′ for example) is present adjacent to the region of breakpoint induction. Other than naturally occurring PAM sites present in genomic DNA, unique PAM sites are generated from single nucleotide polymorphisms, allelic variations and point mutations. Point mutations are extremely common in the cancer mutation landscape and modern genome sequencing technology enables identification of unique patient specific point mutations in various types of cancer. If such point mutations result in a PAM site formation, a CRISPR Cas9 mediated genome editing target is provided within a cancer cell for example, that is not within a normal or healthy nonmutated cell, or a cell otherwise without the specific point mutation.

According to one aspect, the present disclosure contemplates the identification of a random point mutation or mutations in a cancer or cancers by genome sequencing of biopsies of tumor tissue. Any such mutation that generates a PAM for an available Cas9 enzyme site can be targeted with the methods described herein. Such mutations include mutations generating PAM sites inside protein coding regions.

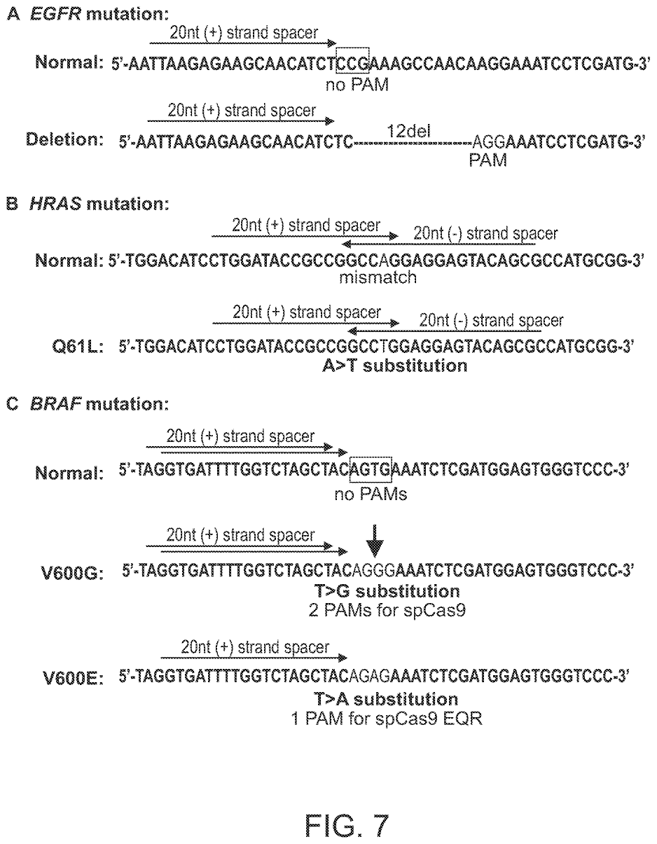

According to the present disclosure, mutations that cause activation or dysregulated expression of oncogenes or activation of a proto-oncogene or inactivation of a tumor suppressor gene are frequently observed in various tumors. These recurrent frequent mutations include a point mutation, small in-frame deletion, single base deletion or substitution, sequence variant generated due to transposon activity, and these events may generate PAM sites, which will enable cancer cell specific mutation targeting. Exemplary oncogenic mutations known to those of skill in the art (see Goon et al., Specific Targeting of Oncogenes Using CRISPR Technology, Cancer Res Oct. 1, 2018 (78) (19) 5506-5512; DOI: 10.1158/0008-5472.CAN-18-0571 that generate a PAM site for CRISPR targeting are shown in FIG. 7 and include deletion of 12 bp in the EGFR gene which causes oncogenic transformation and also generates a novel AGG PAM site present only in the mutant cancer cells. Another exemplary mutation in many types of cancer is a point mutation is HRAS-Q61L. This point mutation generates a novel PAM site as shown in FIG. 7, and sgRNA can be designed based on PAM adjacent sequences from both strands. Similarly point mutations in the BRAF gene, such as a T>G oncogenic point mutation at BRAF codon 600 (V600G) creates several adjacent PAM sequences as shown in FIG. 7. This mutation is also a frequent mutation in melanoma.

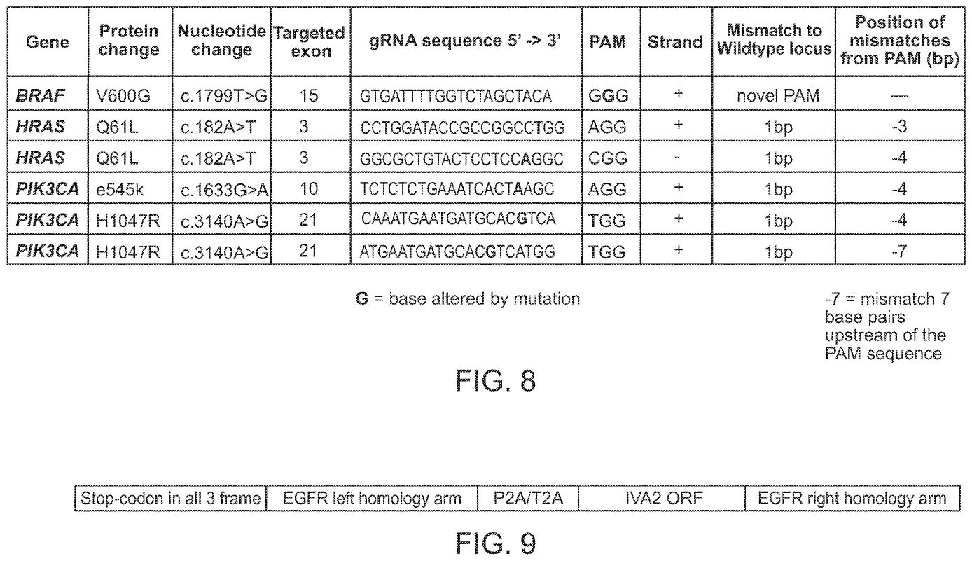

Another point mutation that is frequent in head and neck cancer is a mT>A mutation in BRAF codon 600 (V600E), and that also causes generation of a novel NGAG PAM site, which can be targeted. A few more point-mutations resulting in novel PAM sites are mentioned below. With modern sequencing technology, it is straight forward to find novel PAM sites that are present only in cancer cells for individual patients. Additional mutations are provided in FIG. 8. Additional mutations generating PAM sites are provided in Koo et al., Selective disruption of an oncogenic mutant allele by CRISPR/Cas9 induces efficient tumor regression. Nucleic Acids Res 2017; 45:7897-908.

Unique DNA sequences can also be identified by genome sequencing as described herein. Such unique DNA sequences are known to those of skill in the art or can be determined by routine literature search. Such unique DNA sequences may be generated by chromosomal translocation. Many different choromosomal translocation events associated with cancer have been reported. Chromosomal translocation events result in either overexpression of an oncogene from a breakpoint locus or result in the expression of a fusion protein following the translocation event. Chromosomal translocation events can be targeted with the methods described herein such as when a PAM site is located near a mutation created by the chromosomal translocation event.

An exemplary chromosomal translocation event is Philadelphia chromosome, which results from translocation of DNA fragments between human chromosome 9 and 22. This translocation event causes expression of fusion gene BCR-ABLI which is involved in tyrosine kinase signaling and promotes cell proliferation. ABLI gene is originally located on chromosome 9, and upon translocation it gets fused with the BCR gene on chromosome 22. This translocation event and the resultant fusion gene generates a unique DNA sequence at the point of fusion. See Nowell P C, Hungerford D A. “Chromosome studies on normal and leukemic human leukocytes.” J Natl Cancer Inst (1960). 25:85-100 and Rowley J D. “A new consistent chromosomal abnormality in chronic myelogenous leukemia identified by quinacrine fluorescence and Giemsa staining.” Nature (1973). 243:290-293.

Another common translocation is between chromosome 8 and 14, which is commonly observed in Burkitt's lymphoma. In this example, Myc proto oncogene from chromosome 8 gets translocated to chromosome 14 and causes Myc expression under strong immunoglobulin heavy chain promoter on chromosome 14 resulting in elevated level of Myc expression and uncontrolled cell proliferation. Translocation of DNA fragments between chromosome 8 and 14 results in a unique DNA sequence formation. See Hoffman, Ronald (2009). Hematology: basic principles and practice (PDF) (5th ed.). Philadelphia, PA: Churchill Livingstone/Elsevier. pp. 1304-1305. ISBN 978-0-443-06715-0 and Liu D, Shimonov J, Primanneni S, Lai Y, Ahmed T, Seiter K (2007). “t(8;14;18): a 3-way chromosome translocation in two patients with Burkitt's lymphoma/leukemia”. Mol. Cancer. 6 (1): 35. doi:10.1186/1476-4598-6-35. PMC 1904237. PMID 17547754.

Exemplary translocation mediated gene fusion events reported in cancer include EWSRI in bone- and soft tissue tumors (see Helman et al., Mechanisms of sarcoma development, Nature Reviews Cancer 2003; 3(9):685-694 PMID 12951587) and RET in thyroid carcinomas (see Pierotti, Chromosomal rearrangements in thyroid carcinomas: a recombination or death dilemma, Cancer Letters 2001:166(1): 1-7 PMID 11295280.)

Another exemplary fusion is ETV6-NTRK3 fusion, which is a chromosomal translocation mediated fusion of ETV6 and Neurotrophin 3 receptor gene, which has been described in various types of cancer such as acute myeloid leukemia, infantile fibrosarcoma, mesoblastic nephroma, and breast carcinoma (see Li Z, Tognon C E, Godinho F J, et al: ETV6-NTRK3 fusion oncogene initiates breast cancer from committed mammary progenitors via activation of API complex. Cancer Cell. 12:542-558. 2007 hereby incorporated by reference in its entirety for exemplary fusions).

Another exemplary fusion is TCF3/PBX1 gene fusion, which is common in pre-B acute lymphoblastic leukemia, t(1;19)(q23;p13) which results in a TCF3/PBX1 fusion, and results in a chimeric transcript consisting of two entirely different genes, MEF2D in 1q23 and DAZAPI in 19q13 (see Yuki Y, Imoto I, Imaizumi M, Hibi S, Kaneko Y, Amagasa T, Inazawa J. Identification of a novel fusion gene in a pre-B acute lymphoblastic leukemia with t(1;19)(q23;p13). Cancer Sci. 2004 June; 95(6):503-7. doi: 10.1111/j.1349-7006.2004.tb03240.x. PMID: 15182431; Burmeister T, Gökbuget N, Schwartz S, Fischer L, Hubert D, Sindram A, Hoelzer D, Thiel E. Clinical features and prognostic implications of TCF3-PBX1 and ETV6-RUNX1 in adult acute lymphoblastic leukemia. Haematologica. 2010 February; 95 (2): 241-6. doi: 10.3324/haematol.2009.011346. Epub 2009 Aug. 27. PMID: 19713226; PMCID: PMC2817026; Felice M S, Gallego M S, Alonso C N, Alfaro E M, Guitter M R, Bernasconi A R, Rubio P L, Zubizarreta P A, Rossi J G. Prognostic impact of t(1;19)/TCF3-PBX1 in childhood acute lymphoblastic leukemia in the context of Berlin-Frankfurt-Münster-based protocols. Leuk Lymphoma. 2011 July; 52 (7): 1215-21. doi: 10.3109/10428194.2011.565436. Epub 2011 May 3. PMID: 21534874 each of which is hereby incorporated by reference in its entirety for the teaching of exemplary fusions).

Additional exemplary fusions are described in Mitelman et al., A breakpoint map of recurrent chromosomal rearrangements in human neoplasia, Nature genetics (1997); 15 Spec No: 417-474; PMID 9140409; Mitelman et al. Fusion genes and rearranged genes as a linear function of chromosome aberrations in cancer, Nature Genetics 2004; 36 (4): 331-334 PMID 15054488; and Mitelman et al., Prevalence estimates of recurrent balanced cytogenetic aberrations and gene fusions in unselected patients with neoplastic disorders, Genes, chromosomes & cancer, 2005; 43 (4): 350-366 PMID 15880352.

According to one aspect, the mutation is a point mutation or a frameshift mutation that creates a PAM sequence with an adjacent 5′ protospacer sequence that can be recognized by a guide RNA/CRISPR Cas enzyme colocalization complex. Since the PAM sequence results from a mutation, the wild type sequence adjacent the PAM sequence created by the mutation becomes a 5′ protospacer sequence that is unique to the mutated cell and is not present in the wild-type or non-mutated cell, i.e. because the 5′ protospacer sequence does not have an associated wild type PAM sequence and so could not be targeted by a gRNA for CRISPR activity. Accordingly, a guide RNA can be designed to target the 5′ protospacer sequence and a Cas enzyme will recognize the PAM sequence created by the point mutation and cleave the target nucleic acid sequence. In this manner, the construct can be integrated into the cellular DNA of the target deleterious cell by homologous recombination after the target nucleic acid sequence is cleaved.

According to one aspect, the mutation is one or more point mutations or frameshift mutations that create a unique 5′ protospacer sequence adjacent a wild type PAM sequence that can be recognized by a guide RNA/CRISPR Cas enzyme colocalization complex. Since the 5′ protospacer sequence adjacent the PAM sequence results from the mutation or mutations, the 5′ protospacer sequence is unique to the mutated cell and is not present in the wild-type or non-mutated cell. Accordingly, a guide RNA can be designed to target the protospacer sequence unique to the mutated cell and a Cas enzyme will recognize the PAM sequence and cleave the target nucleic acid sequence. In this manner, the construct can be integrated into the cellular DNA by homologous recombination after the target nucleic acid sequence is cleaved.

According to one aspect, the mutation is a frameshift mutation in a protein coding sequence and the treatment molecule sequence is inserted in frame for the target cell, resulting in expression of the treatment molecule, but out of frame for the non-target cells, resulting in expression of a random non-functional peptide.

The present disclosure also contemplates DNA rearrangements (including e.g. translocations, inversions, insertions, deletions, and duplications) that are found for example in cancer cells due to genomic instability or in immune cells from generating immune cell diversity. These DNA rearrangements which may include a DNA break point can be targeted by designing guide RNAs with spacer sequences complementary to the DNA rearrangement, or adjacent to a PAM sequences created by the DNA rearrangement. Such DNA rearrangements are known to those of skill in the art or can be identified through routine literature search or using genome sequencing technology. See for example, Edwards P A. Fusion genes and chromosome translocations in the common epithelial cancers. J Pathol. 2010 January; 220(2): 244-54. doi: 10.1002/path.2632. PMID: 19921709. According to one aspect, a plurality of Cas enzyme guide RNAs can be designed to overlap the DNA breakpoint at many different locations increasing likelihood that a PAM site is close to the breakpoint and cleaving can take place for integration of the construct.

According to one aspect, a PAM sequence can be generated by viral DNA integration. Many double stranded DNA viruses gets integrated in the genome, often in a tissue or cell specific manner. Such viral integrations may lead to various pathologies including hepatocellular carcinoma to cervical cancer, and also squamous cell carcinomas of neck of head region. Owing to the fact that the integrated viral DNA is completely foreign in nature, and integration is often random or has tissue- or cell-specificity, an aspect of the present disclosure is to utilize unique DNA sequences and PAM sites resulting from the viral DNA integration. Viral DNA integration is associated with the following pathologies. Human papilloma virus (HPV) is associated with cervical cancer and many cases of head and neck squamous cell carcinomas (HNSCC) also result from HPV infection. Integration of HPV DNA in human cellular genomic DNA causes oncogenic transformation of the cells. For example, the E6 gene from HPV promotes degradation of P53 in human cells and E7 from inhibit Retinoblastoma, which is a tumor suppressor. Several PAM sequences have been identified in the HPV genome. These PAM sequences can be used as targets present only in cancer cells and not in cells without the HPV DNA integration, reducing off-target effects. See Cancer Genome Atlas Research Network. Comprehensive genomic characterization of head and neck squamous cell carcinomas. Nature 2015; 517:576-82; zur Hausen H. Papillomaviruses and cancer: from basic studies to clinical application. Nat Rev Cancer 2002; 2:342-50; Hu Z, Ding W, Zhu D, Yu L, Jiang X, Wang X, et al. TALEN-mediated targeting of HPV oncogenes ameliorates HPV-related cervical malignancy. J Clin Invest 2015; 125:425-36; Hu Z, Yu L, Zhu D, Ding W, Wang X, Zhang C, et al. Disruption of HPV16-E7 by CRISPR/Cas system induces apoptosis and growth inhibition in HPV16 positive human cervical cancer cells. BioMed Res Int 2014; 2014:612823. According to one aspect, Epstein-Barr virus (EBV) is associated with pathologic conditions like Burkitt's lymphoma, Hodgkin's lymphoma and nasopharyngeal carcinoma. The role of EBV infection in various types of carcinoma is well known and EBV proteins' role in carcinoma is documented. According to one aspect, EBV DNA itself contains PAM sites or is integrated into the human genome creating PAM sites in the EBV genome or the EBV-human genome boundary. See Desfarges S, Ciuffi A. Viral Integration and Consequences on Host Gene Expression. Viruses: Essential Agents of Life. 2012; 147-175. Published 2012 Sep. 25. doi: 10.1007/978-94-007-4899-6_7; Xu M, Zhang W L, Zhu Q, et al. Genome-wide profiling of Epstein-Barr virus integration by targeted sequencing in Epstein-Barr virus associated malignancies. Theranostics. 2019; 9 (4): 1115-1124. Published 2019 Jan. 30. doi: 10.7150/thno.29622. According to one aspect, PAM sequences generated by hepatitis B virus integration can be targeted using the methods described herein. Integrated hepatitis virus is commonly found in human hepatocellular carcinoma. According to one aspect, hepatitis B viral DNA itself contains PAM sites or is integrated into the human genome creating PAM sites in the EBV genome or the EBV-human genome boundary. See Hino O, Shows T B, Rogler C E. Hepatitis B virus integration site in hepatocellular carcinoma at chromosome 17;18 translocation. Proc Natl Acad Sci USA. 1986 November; 83 (21): 8338-42. doi: 10.1073/pnas.83.21.8338. PMID: 3022290; PMCID: PMC386923.

It is to be understood that the strategies described herein for targeting one or more or a plurality of mutations can also be applied to alternative gene editing technologies as described herein that utilize such mutations or DNA rearrangements as targeting regions for binding to DNA.

According to one aspect, methods of the present disclosure are not limited to mutations creating PAM sites or cleaving by Cas enzymes. The methods of the present disclosure extend to mutations that include DNA translocations and protein fusions which are common in cancer cells for example and provide a unique sequence within a cancer cell to target. For example, a DNA translocation creates a break point. A construct can be integrated into cellular DNA spontaneously by homologous recombination. By targeting a DNA region overlapping with the breakpoint, mutation specific editing is achieved, i.e. the sequence created by the translocation is specific to the mutated cell and does not occur in the wild-type cell. To facilitate integration, the construct includes 5′ and 3′ flanking regions homologous to the target DNA at the desired integration site. For example, a construct including homology arms homologous to the mutated translocation sequence at the breakpoint (and accordingly non-homologous to the wild type sequence) can result in spontaneous integration of the construct. With a mutated translocation sequence, only one homology arm will match for the wild type sequence making off-target integration in the wild type unlikely. It is to be understood, of course, that a nucleic acid construct may include flanking regions for use with a gene editor to facilitate homologous recombination after the cellular DNA is cleaved.

Optionally, to further enhance homologous recombination, alternative DNA repair pathways like non-homologous end joining can be repressed by small RNA, peptides or drugs, e.g. single chain antibodies interfering with Ku protein complex, or Cas9 protein can be fused with homologous recombination proteins. Notably, for DNA rearrangements like translocations, off-target effects due to integration to an incorrect site are extremely unlikely, since only mutant cells have target DNA sites with both flanking homologous regions required for integration by homologous recombination.

An exemplary mutation includes a mutation associate with a cancel cell. An exemplary unique nucleic acid sequence is one associate with an immune cell.

### V. Constructs

According to certain aspects, a nucleic acid construct is a foreign nucleic acid sequence that is exogenous to the cell or non-naturally occurring within the cell (i.e. those which are not part of a cell's natural nucleic acid composition). The foreign nucleic acid sequence may be introduced into a cell using any method known to those skilled in the art for such introduction. Such methods include transfection, transduction, viral transduction, microinjection, lipofection, nucleofection, nanoparticle bombardment, transformation, conjugation and the like. Nucleic acid constructs may be delivered to a subject by administering to the subject, such as systemically administering to the subject, such as by intravenous administration or injection, intraperitoneal administration or injection, intramuscular administration or injection, intracranial administration or injection, intraocular administration or injection, subcutaneous administration or injection, a nucleic acid or vector including a nucleic acid as described herein. One of skill in the art will readily understand and adapt such methods using readily identifiable literature sources. In some embodiments, a construct includes a nucleic acid sequence which is to be inserted into cellular DNA according to methods described herein. The nucleic acid sequence may be expressed by the cell.

According to one aspect, a nucleic acid construct is provided which includes one or more nucleic acids encoding one or more treatment molecules. A plurality of treatment molecules can be encoded by a single nucleic acid construct encoding for each treatment molecule. According to one aspect, a plurality of nucleic acid constructs can be used to target a plurality of mutations that may be within the mutant cell. The nucleic acid construct is designed to discriminate expression of the nucleic acid encoding the treatment molecule or molecules between cells that include the mutation or mutations and cells which lack the mutation or mutations.

According to one aspect, the nucleic acid construct lacks one or more expression elements which thereby inhibits or prevents expression of the nucleic acid encoding the treatment molecule until the nucleic acid construct is integrated into the mutant cell, whereby the cell's expression elements are used to express the nucleic acid encoding the treatment molecule. According to one aspect, the nucleic acid construct includes one or more expression elements that are disrupted or inhibited, and so expression of the nucleic acid encoding the treatment molecule is disrupted, inhibited or prevented until the inhibitor is removed and/or the construct is integrated into the mutant cell.

According to one aspect, expression of the nucleic acid in the construct into RNA or a protein is inhibited or prevented without integration into the target cellular DNA due to incomplete or disrupted transcriptional, translational or splicing elements. For example, the construct can be designed to either lack or disrupt one or more of a promoter, a start codon, a ribosome entry site, a specific splicing sequence, a polyA signal or other mRNA processing signal, a position in-frame of a specific frameshift mutation being targeted after integration or any gene expression element, transcriptional, post-transcriptional or post-translation regulatory sequences, and the like that could otherwise result in protein or RNA production from the nucleic acid encoding the treatment molecule. Such expression of the nucleic acid encoding the treatment molecule is complemented or enabled after integration into cellular DNA at an appropriate target site.

According to one aspect, the nucleic acid construct includes a 5′ flanking nucleic acid and a 3′ flanking nucleic acid, wherein the 5′ flanking nucleic acid and the 3′ flanking nucleic acid are homologous to the target integration site, and wherein the 5′ flanking nucleic acid lacks a gene expression element or includes one or more expression element sufficient for expression of the nucleic acid encoding the treatment molecule but also includes one or more expression inhibition elements, such as a terminal signal or transcription terminator or translation terminator, to inhibit expression of the nucleic acid encoding the treatment molecule.

According to one aspect, the nucleic acid construct is designed to target a mutation in the mutant cell alone or with a gene editor.

### VI. Expression Control Elements

Expression control elements or regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more of a pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signals for introns, 2A peptide sequences (that allow multicistronic expression) and stop codons. The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, which may be referred to as polyA sequences. It also can be designed to enhance mRNA stability. Expression control sequences which affect the transcription and translation stability, e.g., promoters, as well as sequences which effect the translation, e.g., Kozak sequences, suitable for use in insect cells are well known to those skilled in the art. Expression control sequences can be of such nature as to modulate the nucleotide sequence to which it is operably linked such that lower expression levels or higher expression levels are achieved.

“Promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter, including e.g. attenuators or enhancers, but also silencers. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells. The disclosure provides for the operable linking of nucleic acid constructs to a mammalian cell-compatible expression control sequence, e.g., a promoter. Many such promoters are known in the art (see Sambrook and Russell, 2001, supra). Constitutive promoters that are broadly expressed in many cell types, such as the CMV and hEflα promoter are disclosed. Variations of the full-length hEflα are also disclosed which are shorter but still provide effective constitutive expression. Disclosed are promoters that are inducible, tissue-specific, cell-type-specific, or cell cycle-specific.

“3′ UTR” or “3′ non-translated sequence” (also often referred to as 3′ untranslated region, or 3′end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

Expression elements within the scope of the present disclosure are well known to those of skill in the art and include a start codon, a ribosome binding site, a promoter, a splicing sequence, a polyA signal, a mRNA processing signal, a transcriptional regulatory sequence, a post-transcriptional regulatory sequence, or a post-translation regulatory sequence [include list] and the like.

### VII. Expression Inhibition Elements

Aspects of the methods described herein may make use of expression inhibition elements such as stop codons or terminator sequences. A terminator sequence includes a section of nucleic acid sequence that marks the end of a gene or operon in cellular DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein. According to one embodiment, the nucleic acid construct can include one or more of a terminal signal or transcription terminator or translation terminator, such as stop codons, that if removed induces expression of the first nucleic acid encoding the treatment molecule.

### VIII. Tags and Reporter Sequences

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

### IX. Inducible Expression Systems

According to one aspect, expression of the nucleic acid encoding the treatment molecule can be under the influence of an inducible expression system such as an inducible promoter or an inducer-activated enzyme, as is known in the art. Examples include tetracycline or doxycycline inducible gene expression systems. See for example Tight control of gene expression in mammalian cells by tetracycline-responsive promoters. Gossen M & Bujard H. PNAS. 1992 Jun. 15; 89(12): 5547-51.

Additional exemplary inducible expression systems, for example, used in mammalian cells include the Cumate-Controlled inducible gene expression system. This expression system is based on p-cmt and p-cym operon form Pseudomonas putida, and based on the interaction between cumate operator (CuO) and the repressor (CymR). In absence of Cumate, CymR binds to the cumate operator (CuO) DNA element, and when Cumate binds with CymR, it is released from the CuO operator. Similar to a tetracycline operated system, Cumate controlled system can also be engineered as a Cumate-on or Cumate-off system. CymR can be used as an inhibitory factor bound to CuO element, at the upstream of a gene and inhibit RNA polymerase from progressing and transcribing. On Cumate binding, CymR is released from CuO element and allows transcription. CymR can be fused with a transcriptional transactivator, such as VP16, and can be localized to the upstream of a gene by incorporating CuO element in the DNA sequence. In absence of Cumate, presence of VP16, enables transcription. On Cumate addition, CymR binds with the Cumate and the CymR-VP16 fusion protein in released from the DNA, and gene expression is inhibited. See Eaton, R. W. p-Cymene catabolic pathway in Pseudomonas putida F1: Cloning and characterization of DNA encoding conversion of p-cymene to p-cumate. J. Bacteriol. 1997, 179, 3171-3180; Mullick, A.; Xu, Y.; Warren, R.; Koutroumanis, M.; Guilbault, C.; Broussau, S.; Malenfant, F.; Bourget, L.; Lamoureux, L.; Lo, R.; et al. The cumate gene-switch: A system for regulated expression in mammalian cells. BMC Biotechnol. 2006, 6, 43; and Hu M. C., Davidson N. The inducible lac operator-repressor system is functional in mammalian cells. Cell. 1987; 48:555-566. doi: 10.1016/0092-8674 (87) 90234-0.

According to an additional aspect, protein-protein interactions can be used to generate an inducible gene expression system. For example, an inducible expression system can bioengineered from FKBP12 and mTOR. Interaction between FKBP12 (FK506 binding protein 12) and mTOR has been used as an inducible system for gene regulation. Rapamycin and Rapamycin analog FK506 binds to FKBP12 protein, which is present in cell cytoplasm, and activates it to bind with mTOR. Fusion protein of FKBP12 or mTOR with a DNA binding domain (such as the DNA binding domain of ZFHD1), and a transcriptional transactivor can be used as a Rapamycin controlled gene regulatory system. See Rivera, V. M.; Clackson, T.; Natesan, S.; Pollock, R.; Amara, J. F.; Keenan, T.; Magari, S. R.; Phillips, T.; Courage, N. L.; Cerasoli, F., Jr.; et al. A humanized system for pharmacologic control of gene expression. Nat. Med. 1996, 2, 1028-1032; Bierer, B. E.; Mattila, P. S.; Standaert, R. F.; Herzenberg, L. A.; Burakoff, S. J.; Crabtree, G.; Schreiber, S. L. Two distinct signal transmission pathways in T lymphocytes are inhibited by complexes formed between an immunophilin and either FK506 or rapamycin. Proc. Natl. Acad. Sci. USA 1990, 87, 9231-9235; Brown, E. J.; Albers, M. W.; Shin, T. B.; Ichikawa, K.; Keith, C. T.; Lane, W. S.; Schreiber, S. L. A mammalian protein targeted by G1-arresting rapamycin-receptor complex. Nature 1994, 369, 756-758; Sabers, C. J.; Martin, M. M.; Brunn, G. J.; Williams, J. M.; Dumont, F. J.; Wiederrecht, G.; Abraham, R. T. Isolation of a protein target of the FKBP12-rapamycin complex in mammalian cells. J. Biol. Chem. 1995, 270, 815-822; Sabatini, D. M.; Erdjument-Bromage, H.; Lui, M.; Tempst, P.; Snyder, S. H. RAFT1: A mammalian protein that binds to FKBP12 in a rapamycin-dependent fashion and is homologous to yeast TORs. Cell 1994, 78, 35-43; Pomerantz, J. L.; Sharp, P. A.; Pabo, C. O. Structure-based design of transcription factors. Science 1995, 267, 93-96; and Schmitz, M. L.; Baeuerle, P. A. The p65 subunit is responsible for the strong transcription activating potential of NF-kappa B. EMBO J. 1991, 10, 3805-3817.

According to an additional aspect, an abscisic acid controlled gene regulatory system is provided as an inducible gene expression system. Abscisic acid serves as a growth factor in plants and promotes a complex formation between PYLI (abscisic acid receptor) and ABII (protein phosphatase 2C56). Thus, fusing these proteins with a DNA binding element and a transcriptional transactivator can be used as an inducible gene regulatory system, controlled by abscisic acid. See Cutler, S. R.; Rodriguez, P. L.; Finkelstein, R. R.; Abrams, S. R. Abscisic acid: Emergence of a core signaling network. Annu. Rev. Plant. Biol. 2010, 61, 651-679; Gosti, F.; Beaudoin, N.; Serizet, C.; Webb, A. A.; Vartanian, N.; Giraudat, J. ABII protein phosphatase 2C is a negative regulator of abscisic acid signaling. Plant. Cell 1999, 11, 1897-1910; Miyazono, K.; Miyakawa, T.; Sawano, Y.; Kubota, K.; Kang, H. J.; Asano, A.; Miyauchi, Y.; Takahashi, M.; Zhi, Y.; Fujita, Y.; et al. Structural basis of abscisic acid signaling. Nature 2009, 462, 609-614; Yin, P.; Fan, H.; Hao, Q.; Yuan, X.; Wu, D.; Pang, Y.; Yan, C.; Li, W.; Wang, J.; Yan, N. Structural insights into the mechanism of abscisic acid signaling by PYL proteins. Nat. Struct. Mol. Biol. 2009, 16, 1230-1236; Melcher, K.; Ng, L. M.; Zhou, X. E.; Soon, F. F.; Xu, Y.; Suino-Powell, K. M.; Park, S. Y.; Weiner, J. J.; Fujii, H.; Chinnusamy, V.; et al. A gate-latch-lock mechanism for hormone signalling by abscisic acid receptors. Nature 2009, 462, 602-608; and Liang, F. S.; Ho, W. Q.; Crabtree, G. R. Engineering the ABA plant stress pathway for regulation of induced proximity. Sci. Signal. 2011, 4, rs2.

According to an additional aspect, an optogenetically controlled gene expression system is provided as an inducible gene expression system. One of the majorly used optogenetically controlled mammalian gene expression system is based on photo responsive light-oxygen-voltage (LOV) domain-containing protein from Neurospora crassa. When induced by blue light, LOV domain containing proteins dimerize, and expressing LOV domain-transcriptional co-activator is used as an optogenetically controlled gene expression system. Another optogenetically controlled gene regulatory system is based on two plant proteins, Cry2, and CIB1, is also widely used in mammalian cells. See Zoltowski B. D., Crane B. R. Light activation of the LOV protein vivid generates a rapidly exchanging dimer. Biochemistry. 2008; 47:7012-7019. doi: 10.1021/bi8007017; Zoltowski B. D., Schwerdtfeger C., Widom J., Loros J. J., Bilwes A. M., Dunlap J. C., Crane B. R. Conformational switching in the fungal light sensor Vivid. Science. 2007; 316:1054-1057. doi: 10.1126/science.1137128; Wang X., Chen X., Yang Y. Spatiotemporal control of gene expression by a light-switchable transgene system. Nat. Methods. 2012; 9:266-269. doi: 10.1038/nmeth.1892; Wu L., Yang H. Q. CRYPTOCHROME 1 is implicated in promoting R protein-mediated plant resistance to Pseudomonas syringae in Arabidopsis. Mol. Plant. 2010; 3:539-548. doi: 10.1093/mp/ssp107; Yu X., Liu H., Klejnot J., Lin C. The Cryptochrome Blue Light Receptors. Arabidopsis Book. 2010; 8: e0135. doi: 10.1199/tab.0135; Hecht B., Muller G., Hillen W. Noninducible Tet repressor mutations map from the operator binding motif to the C terminus. J. Bacteriol. 1993; 175:1206-1210. doi: 10.1128/jb.175.4.1206-1210.1993.

### X. Systems for Removing Expression Inhibition Elements

According to one aspect, the present disclosure contemplates the use of an enzyme that can remove an expression inhibition element, as is known in the art, so as to activate expression of the nucleic acid encoding the treatment molecule. Exemplary enzymes include an inducible DNA flippase that can remove a transcription or translation terminator or a degradation tag or localization tag flanked by FRT sites, e.g., a stop codon before the nucleic acid encoding the treatment molecule. Drug-inducible DNA flippases have been characterized in the scientific literature. See for example, Feil, R., Wagner, J., Metzger, D., & Chambon, P. (1997). Regulation of Cre Recombinase Activity by Mutated Estrogen Receptor Ligand-Binding Domains. Biochemical and Biophysical Research Communications, 237 (3), 752-757 and Hunter N L, Awatramani R B, Farley F W, Dymecki S M. Ligand-activated Flpe for temporally regulated gene modifications. Genesis. 2005 March; 41 (3): 99-109.

According to an additional example, a Cre recombinase-based system can be used to achieve site specific recombination. Cre recombinase is from Bacteriophages, which recognize the loxP DNA sequence sites. In both FRT-FLP based system, or Cre-loxP based system, a site specific recombination event can trigger the excision or inversion of a nucleotide sequence between two Cre or FRT site. A drug-inducible version of Cre recombinase has been engineered by fusing the ligand binding domain of estrogen receptor with the Cre protein. This form of Cre recombinase can be activated by 4-OH tamoxifen. See Metzger D., Clifford J., Chiba H., Chambon P. Conditional site-specific recombination in mammalian cells using a ligand-dependent chimeric Cre recombinase. Proc. Natl. Acad. Sci. USA. 1995; 92:6991-6995. doi: 10.1073/pnas.92.15.6991; Logie C., Stewart A. F. Ligand-regulated site-specific recombination. Proc. Natl. Acad. Sci. USA. 1995; 92:5940-5944. doi: 10.1073/pnas.92.13.5940; Fuhrmann-Benzakein E., Garcia-Gabay I., Pepper M. S., Vassalli J. D., Herrera P. L. Inducible and irreversible control of gene expression using a single transgene. Nucleic Acids Res. 2000; 28: E99. doi: 10.1093/nar/28.23.e99; Kuhn R., Schwenk F., Aguet M., Rajewsky K. Inducible gene targeting in mice. Science. 1995; 269:1427-1429. doi: 10.1126/science.7660125. Gu H., Marth J. D., Orban P. C., Mossmann H., Rajewsky K. Deletion of a DNA polymerase beta gene segment in T cells using cell type-specific gene targeting. Science. 1994; 265:103-106. doi: 10.1126/science.8016642; Abremski K., Hoess R. Bacteriophage P1 site-specific recombination. Purification and properties of the Cre recombinase protein. J. Biol. Chem. 1984; 259:1509-1514; Cox M. M. The FLP protein of the yeast 2-microns plasmid: Expression of a eukaryotic genetic recombination system in Escherichia coli. Proc. Natl. Acad. Sci. USA. 1983; 80:4223-4227. doi: 10.1073/pnas.80.14.4223; and Hoess R. H., Ziese M., Sternberg N. P1 site-specific recombination: Nucleotide sequence of the recombining sites. Proc. Natl. Acad. Sci. USA. 1982; 79:3398-3402. doi: 10.1073/pnas.79.11.3398.

According to an additional example, recombination mediated cassette exchange can accomplish incorporation of a specific DNA sequence at a specific site, and can be used as a trigger or suppressor for gene expression. Such RMCE events can be triggered by FRT mediated cassette exchange or attP, attB mediated cassette exchange. See Turan S, Zehe C, Kuehle J, Qiao J, Bode J. Recombinase-mediated cassette exchange (RMCE)—a rapidly-expanding toolbox for targeted genomic modifications. Gene. 2013 Feb. 15; 515 (1): 1-27. doi: 10.1016/j.gene.2012.11.016. Epub 2012 Nov. 29. Erratum in: Gene. 2013 May 10; 520 (1): 77. PMID: 23201421.

### XI. Vectors for Delivery of the Construct

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, double stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleic acid sequence (e.g. in an in vitro transcription/translation system or in a host cell when the vector is introduced into the host cell).

The term vector also includes methods of non-viral delivery of nucleic acids or native DNA binding protein, native guide RNA or other native species including lipofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

According to one aspect, the nucleic acid construct including the nucleic acid encoding the treatment molecule is within a vector that is used to deliver the construct described herein. Exemplary vectors include a virus, a phage, a liposome, a microorganism, a nanoparticle and the like.

In some embodiments, the vectors for use in the methods herein are parvoviral vectors, such as animal parvoviruses, in particular dependoviruses such as infectious human or simian adeno-associated virus (AAV), and the components thereof (e.g., an animal parvovirus genome) for use as vectors for introduction and/or expression of the nucleotide sequences encoding a porphobilinogen deaminase in mammalian cells. Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, and 6) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth 1. Berns, “Parvoviridae: The Viruses and Their Replication,” Chapter 69 in Fields Virology (3d Ed. 1996). For convenience the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes (i.e., Rep78 and Rep52) are expressed from the P5 promoter and the P19 promoter, respectively and both Rep proteins have a function in the replication of the viral genome. A splicing event in the Rep ORF results in the expression of actually four Rep proteins (i.e., Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

A “recombinant parvoviral” or “AAV vector” or “rAAV vector” herein refers to a vector comprising one or more polynucleotide sequences of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions. Thus, in a further aspect the invention relates to a nucleic acid construct comprising a nucleotide sequence encoding a porphobilinogen deaminase as herein defined above, wherein the nucleic acid construct is a recombinant parvoviral or AAV vector and thus comprises at least one parvoviral or AAV ITR. Preferably, in the nucleic acid construct the nucleotide sequence encoding the porphobilinogen deaminase is flanked by parvoviral or AAV ITRs on either side.

AAV is able to infect a number of mammalian cells. See, e.g., Tratschin et al., (1985) Mol. Cell Biol. 5:3251-3260) and Grimm et al., (1999) Hum. Gene Ther. 10:2445-2450). However, AAV transduction of human synovial fibroblasts is significantly more efficient than in similar murine cells, (Jennings et al., (2001) Arthritis Res, 3:1), and the cellular tropicity of AAV differs among serotypes. See, e.g., Davidson et al. (2000) Proc. Natl. Acad. Sci. USA, 97:3428-3432), which discuss differences among AAV2, AAV4, and AAV5 with respect to mammalian CNS cell tropism and transduction efficiency; Goncalves, (2005) Virol J. 2 (1): 43, which discusses approaches to modification of AAV tropism. In some embodiments, for transduction of liver cells rAAV virions with AAV1, AAV8 and AAV5 capsid proteins are preferred (Nathwani et al., (2007) Blood 109 (4): 1414-1421; Kitajima et al., (2006) Atherosclerosis 186 (1): 65-73), of which is rAAV virions with AAV5 capsid proteins may be most preferred.

AAVs are highly prevalent within the human population. See Gao, G., et al., (2004) J Virol. 78 (12): 6381-8; and Boutin, S., et al., (2010) Hum Gene Ther. 21 (6): 704-12) and are useful as viral vectors. Many serotypes exist, each with different tropism for tissue types, See Zincarelli, C., et al., (2008) Mol Ther. 16 (6): 1073-80), which allows specific tissues to be preferentially targeted with appropriate pseudotyping. Some serotypes, such as serotypes 8, 9, and rh10, transduce the mammalian body. See Zincarelli, C., et al., (2008) Mol Ther. 16 (6): 1073-80, Inagaki, K., et al., (2006) Mol Ther. 14 (1): 45-53; Keeler, A. M., et al., (2012) Mol Ther. 20(6):1131-8; Gray, S. J. et al., (2011) Mol Ther. 19 (6): 1058-69; Okada, H., et al., (2013) Mol Ther Nucleic Acids. 2: e95; and Foust, K. D., et al., (2009) Nat Biotechnol. 27 (1): 59-65. AAV9 has been demonstrated to cross the blood-brain barrier. See Foust, K. D., et al., (2009) Nat Biotechnol. 27 (1): 59-65; and Rahim, A. A. et al., (2011) FASEB J. 25 (10): 3505-18) that is inaccessible to many viral vectors and biologics. Certain AAVs have a payload of 4.7-5.0 kb, including viral inverted terminal repeats (ITRs), which are required in cis for viral packaging). See Wu, Z. et al., (2010) Mol Ther. 18 (1): 80-6; and Dong, J. Y. et al., (1996) Hum Gene Ther. 7 (17): 2101-12; all publications incorporated herein by reference.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped rAAV particles comprising the capsid proteins of one serotype (e.g., AAV5) and the Rep and/or ITR sequences of another AAV serotype (e.g., AAV2). Such pseudotyped rAAV particles are a part of the present invention. Herein, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV5. Modified “AAV” sequences also can be used in the context of the present disclosure, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having from about 75% to about 99% nucleotide sequence identity) to an AAV1, AAV2, AAV3, AAV4, AAV5, AAV6, AAV7, AAV8, AAV9, AAV 10, AAV11, AAV12, AAV2.5, AAvDJ, AAVrh10.XX ITR, Rep, or VP can be used in place of wild-type AAV ITR, Rep, or VP sequences. Preferred adenoviral vectors are modified to reduce the host response. See, e.g., Russell (2000) J. Gen. Virol. 81:2573-2604; US patent publication no. 20080008690; and Zaldumbide et al. (2008) Gene Therapy 15 (4): 239-46; all publications incorporated herein by reference.

According to one aspect the vector is a viral vector, such as an adenovirus vector, such as Ad5, Ad26, Ad35, and Ad38 and the like. Adenoviruses (members of the family Adenoviridae) are known in the art and are medium-sized (90-100 nm), nonenveloped (without an outer lipid bilayer) viruses with an icosahedral nucleocapsid containing a double stranded DNA genome. Adenoviruses are known to be useful vectors. See for example Rapid Cloning of Novel Rhesus Adenoviral Vaccine Vectors DOI: 10.1128/JVI.01924-17).

According to one aspect, the vector is a herpes simplex virus. For example, Herpes simplex virus can be used as a gene delivery agent, as described in Burton E A, Fink D J, Glorioso J C. Gene delivery using herpes simplex virus vectors. DNA Cell Biol. 2002 December; 21 (12): 915-36. doi: 10.1089/104454902762053864. PMID: 12573050; Burton E A, Wechuck J B, Wendell S K, Goins W F, Fink D J, Glorioso J C., Multiple applications for replication-defective herpes simplex virus vectors. Stem Cells. 2001; 19 (5): 358-77. doi: 10.1634/stemcells.19-5-358.PMID: 11553845; Lachmann, Herpes simplex virus-based vectors. R. Int J Exp Pathol. 2004 October; 85 (4): 177-90. doi: 10.1111/j.0959-9673.2004.00383.x.PMID: 15312123; Huard J, Goins W F, Glorioso J C. Herpes simplex virus type 1 vector mediated gene transfer to muscle. Gene Ther. 1995 August; 2 (6): 385-92.PMID: 7584113.

According to one aspect, the virus vector replicates within the target mutated cell.

According to one aspect, the virus vector excludes a gene required for replication and includes a third nucleic acid encoding the gene required for replication, wherein the third nucleic acid is inserted into the cellular DNA of the target mutated cell, wherein the third nucleic acid encoding the gene required for replication is expressed, and the virus replicates within the target mutated cell.

According to one aspect, the vector lacks one or more expression elements so as to inhibit or prevent expression of the nucleic acid encoding the treatment molecule before the nucleic acid construct is integrated into the cellular DNA.

Methods of delivering nucleic acids, such as genes to subjects, for example using adeno-associated viruses, are described in U.S. Pat. No. 6,967,018, WO2014/093622, US2008/0175845, US 2014/0100265, EP2432490, EP2352823, EP2384200, WO2014/127198, WO2005/122723, WO2008/137490, WO2013/142114, WO2006/128190, WO2009/134681, EP2341068, WO2008/027084, WO2009/054994, WO2014059031, U.S. Pat. No. 7,977,049 and WO 2014/059029, each of which are incorporated herein by reference in its entirety.

### XII. Exemplary Genes

Exemplary genes required for replication of adenovirus include encapsidation protein Iva2 or control protein E1A and the like. Other Ad5 viral proteins thought to be essential for replication include capsid protein IX, DNA polymerase, protein 13.6K, terminal protein precursor pTP, encapsidation protein 52K, capsid protein precursor pIlla, penton base (capsid protein III), core, protein precursor pVII, core protein V, core protein precursor pX, capsid protein precursor pVI, hexon (capsid protein II), protease, single-stranded DNA-binding protein, hexon assembly, protein 100K, protein 33K, encapsidation protein 22K, capsid protein precursor pVIII, protein U, fiber (capsid protein IV), control protein E4orf3 or E4orf6.

According to one aspect, genes that are non-essential for replication, but enhance replication can be used. Such genes include control protein EIB 19K, control protein EIB 55K, control protein, E3 12.5K, membrane glycoprotein E3 CR1-alpha, membrane glycoprotein E3 gp19K, membrane, glycoprotein E3 CR1-beta, membrane glycoprotein E3 CR1-delta, membrane protein, E3 RID-alpha, membrane protein E3 RID-beta, control protein E3 14.7, control protein E4 34K, control protein E4orf4, control protein E4orf3, control protein E4orf2, control protein E4orfl and either control protein E4orf3 or control protein E4orf6 (complemented by the presence of the other gene).

Exemplary genes associated with other viral vectors include virion host shutoff protein (VHS or UL41) in herpes simplex virus, which plays an important role for viral replication (see Miles C Smith, Chris Boutell, and David J Davido, HSV-1 ICPO: paving the way for viral replication, Future Virology 2011 6:4, 421-429), UL5 protein important for viral DNA replication and UL30 encoding DNA polymerase (see Duncan J. McGeoch, Frazer J. Rixon, Andrew J. Davison, Topics in herpesvirus genomics and evolution, Virus Research, Volume 117, Issue 1, 2006, Pages 90-104.)

Another example of a protein important for viral propagation in herpes simplex virus is RS1, a major transcriptional activator essential for progression of viral infection beyond initial infection stages. See Wagner L M, Bayer A, Deluca N A. Requirement of the N-terminal activation domain of herpes simplex virus ICP4 for viral gene expression. J Virol. 2013 January; 87 (2): 1010-8. doi: 10.1128/JVI.02844-12. Epub 2012 Nov. 7. PMID: 23135715; PMCID: PMC3554072.

Another example is UL6 in herpes simplex virus, encoding twelve proteins constituting the capsid portal ring through which DNA enters and exits the capsid. See Wagner L M, Bayer A, Deluca N A. Requirement of the N-terminal activation domain of herpes simplex virus ICP4 for viral gene expression. J Virol. 2013 January; 87 (2): 1010-8. doi: 10.1128/JVI.02844-12. Epub 2012 Nov. 7. PMID: 23135715; PMCID: PMC3554072.

It is to be understood that the above examples are not exhaustive and that one of skill can identify by literature search similar such genes associated with replication and propagation of infection, as are known to those of skill in the art.

### XIII. Target Integration Sites

According to one aspect, expression of the nucleic acid encoding the treatment molecule such as a protein or RNA by target cells occurs only after integration into target cellular DNA. To achieve this, the integration site in the target cellular DNA is selected to complement the missing expression element or elements nucleic acid encoding the treatment molecule. For example, protein or RNA expression can be enabled by integration in a position
a. downstream from a promoter or essential promoter element
b. downstream from a transcription start site
c. downstream from a start codon
d. downstream from a ribosome entry site
e. in frame with a targeted frameshift mutation
f. for specific splicing sites
g. for a polyA signal
h. with respect to any gene expression element, transcriptional, post-transcriptional or post-translation regulatory sequences (e.g. splicing, protein modifications or cleavage, degradation or inhibition of repressors) that will enable cargo RNA or cargo protein expression or accumulation after integration of cargo DNA (but which are lacking or disrupted in the cargo DNA before integration or when cargo DNA is integrated into DNA lacking the targeted mutation).

According to one aspect, self-cleaving peptides can be used to ensure correct folding and functionality of delivered proteins if fusion proteins are generated by the integration strategy. For example, when start codons in targeted DNA are used for integration-specific protein expression, a self-cleaving peptide can split the fusion of the protein expressed from the target DNA away from the cargo protein. Self-cleaving peptides can also be used to express several proteins after correct integration to target sites by expression of all the desired proteins in one fusion protein separated by self-cleaving peptides that subsequently cleave the fusion protein in the desired products.

### XIV. Administration Dosage and Treatment

In some embodiments, a therapeutically effective amount of a construct including the nucleic acid encoding the treatment molecule is administered to the subject to treat deleterious cells. In various embodiments, the one or more vectors, including viral vectors, and packaged viral particles containing the viral vectors, can be in the form of a medicament or a pharmaceutical composition and may be used in the manufacture of a medicament or a pharmaceutical composition. The pharmaceutical composition may include a pharmaceutically acceptable carrier. Preferably, the carrier is suitable for parenteral administration. In particular embodiments, the carrier is suitable for intravenous, intraperitoneal or intramuscular administration. Pharmaceutically acceptable carrier or excipients are described in, for example, Remington: The Science and Practice of Pharmacy, Alfonso R. Gennaro (Editor) Publishing Company (1997). Exemplary pharmaceutical forms can be in combination with sterile saline, dextrose solution, or buffered solution, or other pharmaceutically acceptable sterile fluids. Alternatively, a solid carrier, may be used such as, for example, microcarrier beads.

Pharmaceutical compositions are typically sterile and stable under the conditions of manufacture and storage. Pharmaceutical compositions may be formulated as a solution, microemulsion, liposome, or other ordered structure suitable to delivery of the gene therapy vectors. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. The vectors of the present disclosure may be administered in a time or controlled release formulation, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymers (PLG).

In some embodiments, the vectors, formulated with any acceptable carriers, can be administered parenterally, such as by intravenous, intraperitoneal, subcutaneous, intramuscular administration, limb perfusion or combinations thereof. The administration can be systemic, such that the vectors are delivered through the body of the subject. In some embodiments, the vectors can be administered directly into the targeted tissue, such as to the heart, liver, synovium, or intrathecally for neural tissues. In some embodiments, the vectors can be administered locally, such as by a catheter. The route of administration can be determined by the person of skill in the art, taking into consideration, for example, the nature of target tissue, vectors, intended therapeutic effect, and maximum load that can be administered and absorbed by the targeted tissue(s).

Generally, an effective amount, particularly a therapeutically effective amount, of the gene delivery vectors are administered to a subject in need thereof. A “therapeutically effective amount” refers to an amount effective, at dosages and for periods of time necessary, to achieve the desired therapeutic result, such as treatment of deleterious cells in a manner to kill such cells. An effective or therapeutically effective amount of vector may vary according to factors such as the disease state, age, sex, and weight of the individual, and the ability of the viral vector to elicit a desired response in the individual. Dosage regimens may be adjusted to provide the optimum therapeutic response.

In particular embodiments, a range for therapeutically or prophylactically effective amounts of a nucleic acid, nucleic acid construct, parvoviral virion or pharmaceutical composition may be from 1×10¹¹ and 1×10¹⁴ genome copy (gc)/kg or 1×10¹² and 1×10¹³ genome copy (gc)/kg. It is to be noted that dosage values may vary with the severity of the condition to be alleviated. The dosage may also vary based on the efficacy of the virion employed. For example AAV8 is better at infecting liver as compared to AAV2 and AAV9 is better at infecting brain than AAV8, in these two cases one would need less AAV8 or AAV9 for the case of liver or brain respectively. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners.

The tissue target may be specific or it may be a combination of several tissues. Exemplary tissue targets may include liver, skeletal muscle, heart muscle, adipose deposits, kidney, lung, vascular endothelium, epithelial and/or hematopoietic cells. In some embodiments, the effective dose range for small animals (mice), following intramuscular injection, may be between 1×10¹² and 1×10¹³ genome copy (gc)/kg, and for larger animals (cats or dogs) and for human subjects, between 1×10¹¹ and 1×10¹² gc/kg, or between 1×10¹¹ and 1×10¹⁴ genome copy (gc)/kg.

In various embodiments, the vectors can be administered as a bolus or by continuous infusion over time. In some embodiments, several divided doses can be administered over time or the dose may be proportionally reduced or increased as indicated by the exigencies of the therapeutic situation. In some embodiments, the gene delivery vectors can be administered daily, weekly, biweekly or monthly. The duration of treatment can be for at least one week, one month, 2 months, 3 months, 6 months, or 8 month or more. In some embodiments, the duration of treatment can be for up to 1 year or more, 2 years or more, 3 years or more or indefinitely.

### XV. Cellular DNA Editors

Aspects of the present disclosure contemplate the use of a nucleic acid editor to cleave cellular DNA in a mutation specific manner or unique DNA sequence manner to facilitate integration of the nucleic acid delivery agent. Such nucleic acid editors are well known to those of skill in the art and include a CRISPR enzyme, a TALENS, a zinc-finger nuclease and a restriction enzyme.

### A. Type II CRISPR Systems

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (SEQ ID NO:14) (see H. Deveau et al., Phage response to CRISPR-encoded resistance in Streptococcus thermophilus. Journal of bacteriology 190, 1390 (February 2008) hereby incorporated by reference in its entirety), respectively, while different S. mutans systems tolerate NGG or NAAR (SEQ ID NO:15) (see J. R. van der Ploeg, Analysis of CRISPR in Streptococcus mutans suggests frequent occurrence of acquired immunity against infection by M102-like bacteriophages. Microbiology 155, 1966 (June 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLOS genetics 8, e1002441 (2012) and D. T. Pride et al., Analysis of streptococcal CRISPRs from human saliva reveals substantial sequence diversity within and between subjects over time. Genome research 21, 126 (January 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes MI GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csnl. An exemplary S. pyogenes Cas9 protein sequence is provided in Deltcheva et al., Nature 471, 602-607 (2011) hereby incorporated by reference in its entirety.

Modification to the Cas9 protein is contemplated by the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

According to one aspect, Cas9 enzymes with modified PAM sites will be utilized. Such enzymes have been generated by mutagenesis and selection. See for example Kleinstiver, B., Prew, M., Tsai, S. et al. Engineered CRISPR-Cas9 nucleases with altered PAM specificities. Nature 523, 481-485 (2015).

An exemplary CRISPR system includes the S. thermophiles Cas9 nuclease (STI Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M. L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art. The Cas9 protein complexed with the guide RNA, known as a ribonucleotide protein (RNP) complex, may also be introduced to the cells via electroporation, injection, or lipofection.

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to one aspect, the guide RNA is between about 100 to about 500 nucleotides. According to one aspect, the guide RNA is between about 100 to about 250 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

According to one aspect, Cas enzymes other than Cas9 may be used in the present methods. Such enzymes have nuclease activity but alternative DNA targeting mechanisms. For example CRISPR-Cpf1, which is an alternative RNA-guided endonuclease from Acidaminococcus sp. (AsCpf1) and Lachnospiraceae bacterium (LbCpf1), do not require any tracrRNA and recognizes 5′-TTN-3′ or 5′-TTTV-3′ (SEQ ID NO:16) as a PAM, as compared to 5′-NGG-3′ PAM identified by Sp Cas9. Additional enzymes known to those of skill use 5′TYCV3′ (SEQ ID NO:17) and 5′TATV3′ (SEQ ID NO:18) as PAMs. See Zetsche et al., Cpf1 is a single RNA-guided endonuclease of a class 2 CRISPR-Cas system. Cell 2015; 163:759-71; Gao et al., Engineered Cpf1 variants with altered PAM specificities. Nat Biotechnol 2017; 35:789-92. Accordingly, aspects of the present disclosure contemplate use of a Cas enzyme of a Type II CRISPR system.

### B. TALEN Systems

According to one aspect, a TALEN as is known in the art is contemplated as the gene editor system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. See Boch, Nature Biotechnology, 29 (2): 135-6 (2011) hereby incorporated by reference in its entirety for the teaching of TALENS and how they work. The restriction enzymes can be introduced into cells, for use in gene editing or for genome editing in situ, a technique known as genome editing with engineered nucleases.

TAL effectors are proteins that are secreted by Xanthomonas bacteria via their type III secretion system when they infect plants. The DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This straightforward relationship between amino acid sequence and DNA recognition has allowed for the engineering of specific DNA-binding domains by selecting a combination of repeat segments containing the appropriate RVDs. Notably, slight changes in the RVD and the incorporation of “nonconventional” RVD sequences can improve targeting specificity.

The non-specific DNA cleavage domain from the end of the FokI endonuclease can be used to construct hybrid nucleases that are active in a yeast cells, plant cells and animal cells. Either the wild-type FokI cleavage domain or variants may be used as is known in the art for cleavage specificity and cleavage activity. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites are parameters for achieving activity.

The relationship between amino acid sequence and DNA recognition of the TALE binding domain allows for the engineering of proteins. According to one aspect, a publicly available software program (DNAWorks[20]) to calculate oligonucleotides is suitable for assembly in a two step PCR oligonucleotide assembly followed by whole gene amplification. A number of modular assembly schemes for generating engineered TALE constructs have been reported. Both methods offer a systematic approach to engineering DNA binding domains that is conceptually similar to the modular assembly method for generating zinc finger DNA recognition domains.

Once the TALEN constructs have been assembled, they are inserted into plasmids; the target cells are then transfected with the plasmids, and the gene products are expressed and enter the nucleus to access the genome. Alternatively, TALEN constructs can be delivered to the cells as mRNAs, which removes the possibility of genomic integration of the TALEN-expressing protein. Using an mRNA vector can also dramatically increase the level of homology directed repair (HDR) and the success of introgression during gene editing.

TALEN can be used to edit genomes by inducing double-strand breaks (DSB), which cells respond to with repair mechanisms. The constructs described herein can be introduced at the double stranded break as templates for cellular repair enzymes.

### C. Zinc Finger Nucleases

As is known in the art, zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms.

The DNA-binding domains of individual ZFNs typically contain between three and six individual zinc finger repeats and can each recognize between 9 and 18 base pairs. If the zinc finger domains perfectly recognize a 3 base pair DNA sequence, they can generate a 3-finger array that can recognize a 9 base pair target site. Other procedures can utilize either 1-finger or 2-finger modules to generate zinc-finger arrays with six or more individual zinc fingers. As with TALENS, the non-specific DNA cleavage domain from the end of the FokI endonuclease can be used.

### D. Restriction Enzymes

As is known in the art, restriction enzymes are naturally occurring or modified enzymes that have the ability to cut nucleic acids. Exemplary restriction enzymes include, for example, AbaSI, AcuI, AhdI, Bael, BcgI, BpmI, BpuEI, BsaXI, CspCI, I-SecI, I-CeuI, PI-SecI, PI-PspI, and the like. One of skill can identify further exemplary useful restriction enzymes through literature searches and at New England Biolabs, world wide website neb.com/tools-and-resources/seelction-charts/frequencies-of-restriction-sites.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Preparation of an Adenoviral Transfer Plasmid (Mcherry Cas Plasmid), and Delivery by Electroporation in HCI1975 and HCI1650 Cells

A single oncogenic point mutation was targeted in EGFR that is very commonly found in cancers. Cell line NCI H1975 is a lung cancer epithelial line that harbors this oncogenic point mutation in EGFR. NCI H1650 is a different lung epithelial cancer cell line with a non-mutated wild type EGFR gene. Delivery of a fluorescent protein in red (mcherry) dependent on the presence of this specific point mutation was demonstrated.

An Ad5 adenovirus vector was used to deliver a cargo gene encoding mcherry, where the start codon, the ribosome binding site and the promoter were removed from the cargo gene and replaced with an in-frame 500 bp region homologous to EGFR followed by a self-cleaving peptide sequence (T2A). The left EGFR homologous region was modified to delete any in-frame start codons that could result in leaky expression from the vector. The other side of the mcherry gene was also flanked by a 500 bp homologous region with EGFR. This design ensures that no cargo protein is expressed from the vector without integration into the chromosome.

To facilitate integration into the chromosome, with the same vector, a nucleic acid encoding Cas9 was delivered under an EF-alpha promoter followed by an in-frame self-cleaving peptide (P2A) and GFP to track the transfection efficiency and the expression of Cas9.

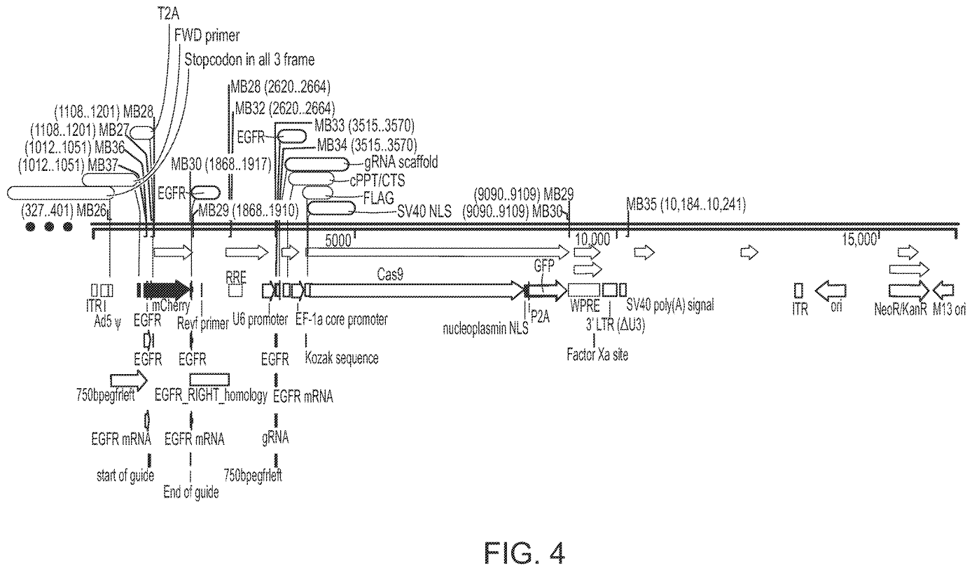

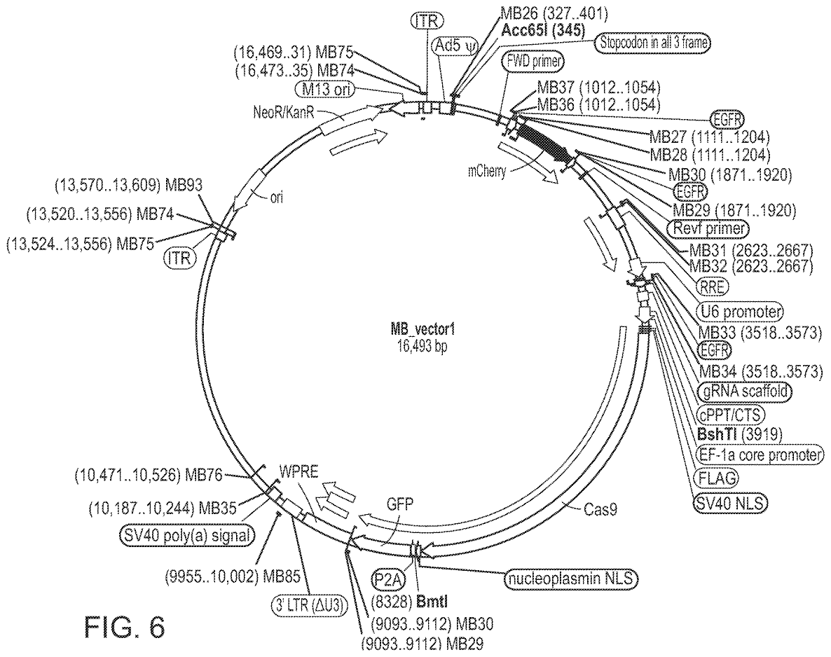

Also in the same vector, a nucleic acid encoding a guide RNA under a U6 promoter was delivered. The oncogenic point mutation in EGFR forms a Cas9 PAM site that is essential for cutting by Cas9. This results in point mutation-specific cutting EGFR DNA in H1975, but not in H1650 or wild type cells. FIG. 4 and FIG. 6 describe the plasmid architecture.

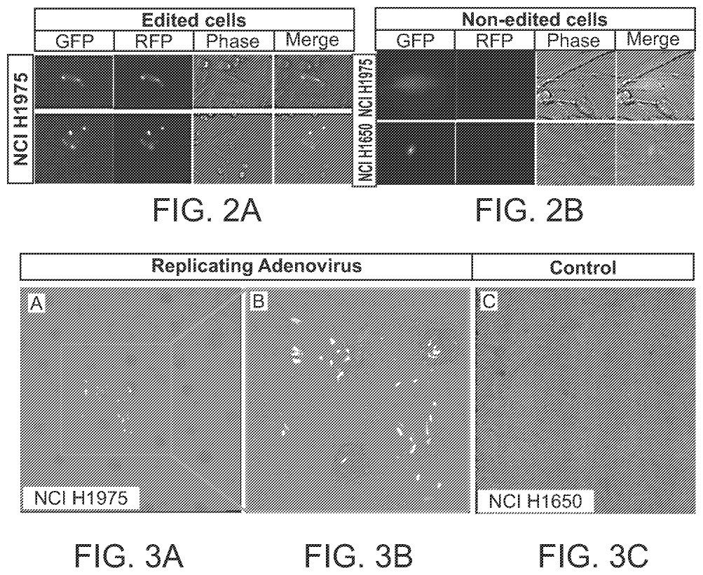

With reference to FIGS. 2A and 2B, roughly 50% of transfected H1975 cells started expressing mcherry, indicating successful editing and gene delivery. On the other hand, no expression of mcherry was found in H1650 cells, despite transfection of the construct, demonstrating mutation specific delivery and expression of the cargo gene.

According to one aspect, the mcherry cargo gene is replaced by extremely potent toxin genes, essentially turning each edited cancer cell into a small toxin factory. With the production of the toxin, each toxin producing cell will not only destroy itself, but also many neighboring cancer cells as the toxin leaks out.

The following describes detailed experimental protocols.

The following cell lines were used: NCI-H1650 [H-1650, H1650] ATCC catalog number: CRL-5883, NCI-H1975 [H-1975, H1975] ATCC catalog number: CRL-5908 were obtained from ATCC.

The following cell culture protocol was used: H1650 and H1975 cells are cultured in RPMI1650 media (Corning) supplemented with 10% bovine growth serum (Gibco), GlutMax (Gibco) and antibiotic-antimicotic cocktail (anti-anti, Gibco). Cell lines are grown in standard T25 or T75 cell culture flasks (BD) and maintained in a humidified incubator (Thermo Scientific) set at 37° C. with 5% carbon dioxide.

The Mcherry Cas9 plasmid is prepared as follows and includes the following elements.
Plasmid backbone: pShuttle (Addgene plasmid 16402)

Insert elements: 750 bp region of the EGFR gene, serves as a left homology arm during homology dependent repair, downstream to that a T2A self-cleaving peptide, followed by mCherry with membrane localization signal, followed by 750 bp region from the EGFR gene, serves as the right homology arm during homology dependent repair. Downstream, the plasmid expresses single guide RNA (sgRNA) targeting the specific point mutation in EGFR gene. Further downstream in this plasmid, EF1-alpha promoter drives the expression of Cas9: Self cleaving peptide P2A: GFP.

| Cell line | EGFR gene region of interest (SEQ ID NO:19-20) |
|---|---|
| H1975 | gtcaagatcacagattttgggcGgg → Point mutation G resulted in PAM site |
| H1650 | gtcaagatcacagattttgggctgg → No PAM site present when t is present |

Mcherry Cas9 plasmid was constructed as a transfer plasmid (for cargo delivery), which can be incorporated into adenoviral genome. To produce adenovirus, three different methods are used: (1) the AdEasy method developed by Bert Vogelstein (He T C, Zhou S, da Costa L T, Yu J, Kinzler K W, Vogelstein B. A simplified system for generating recombinant adenoviruses. Proc Natl Acad Sci USA. 1998 Mar. 3; 95 (5): 2509-14. doi: 10.1073/pnas.95.5.2509. PMID: 9482916; PMCID: PMC19394.); (2) a strategy of adenovirus production described by Miciak et. Al. (Miciak J J, Hirshberg J, Bunz F (2018) Seamless assembly of recombinant adenoviral genomes from high-copy plasmids. PLOS ONE 13 (6): e0199563. doi.org/10.1371/journal.pone.0199563, referred to as Adenobuilder; (3) a strategy described by Miciak J J et. al., PLOS ONE 13 (6): e0199563 for changing any location on the adenovirus genome using NeBuilder DNA assembly, but producing full adenovirus genomic DNA in E. coli before transfection as in the tradition AdEasy approach.

Three different versions of the transfer plasmid were prepared to make it compatible with three different strategies. To make it compatible with the AdEasy strategy, pShuttle was used as the plasmid backbone, and DNA fragments (as described in plasmid architecture above) were clones into the pShuttle backbone by NeBuilder DNA assembly. This plasmid (Mcherry Cas9 plasmid) can directly be used to recombine with AdEasy plasmids to reconstitute the adenoviral genome following the AdEasy protocol.

To make this transfer plasmid compatible with the adenovirus preparation method described by Miciak J J et. Al., the Mcherry Cas9 plasmid was further modified by inserting two BstBI sites flanking the viral and construct DNA, while removing the homologous region required for the AdEasy strategy, and replacing it with a short overlap required for NeBuilder DNA assembly. In the strategy by Miciak J J et. al., adenovirus genome is distributed over seven plasmids named pAd5B1 to pAd5B7. For the seven plasmids, pAd5B1 serves as the transfer plasmid for cargo DNA and other six plasmids contain essential elements of Ad5 genome. In each plasmid, adenoviral DNA component is flanked by two BstBI restriction enzyme sites. As a result, digestion with BstB1 yields a linear double stranded fragment from each plasmid and these double stranded fragments can be assembled together by NeBuilder DNA assembly due to the presence of overlapping homology sequence among them. The full linear adenoviral genome assembled in this way can be transfected into mammalian cells, where they can initiate viral replication and be packaged into viral capsids.

The strategy described by Miciak J J et. Al., is altered by cloning the assembled adenoviral genome from pAd5 plasmids in a single plasmid in order to store assembled constructs long-term and to increase the amount of DNA production before transfection. The altered version of Mcherry Cas9 plasmid that is compatible with NeBuilder DNA assembly of a single plasmid containing the adenovirus genome for replication in E. coli is named workhorse plasmid and it contains single BstBI site. This plasmid can be seamlessly assembled with other adenoviral fragments from adenobuilder pAd5 plasmids to contain the adenoviral genome, as well as the desired construct in one single plasmid. After DNA production in E. coli, the backbone of this large plasmid can be separated from the adenovirus genome by PacI digestion and transfected into mammalian cells (just as in the AdEasy strategy).

Mcherry Cas9 plasmid was prepared by cloning DNA fragments in pShuttle plasmid backbone (Addgene plasmid 16402) by NeBuilder 2× HiFi DNA assembly master mix (New England Biolabs, M5520AA) DNA assembly method, following the manufacturer's protocol. All double stranded DNA fragments were amplified by Q5-2× high fidelity hot start DNA polymerase master mix (NEB, M0494S), unless otherwise mentioned. For polymerase chain reaction, the manufacturer (NEB) recommended protocol was followed, unless otherwise mentioned. All primers are synthesized through IDT or Genewiz as single stranded oligo with standard desalting, unless otherwise mentioned.

EGFR left homology arm was synthesized as a double stranded gene block (from Twist biosciences) and stop codons in all three frames were added to completely eliminate any possibility of leaky protein expression without chromosomal integration from the beginning of the double stranded gene block. For the same reason, all ATG triplets were replaced within these 750 base pair regions. In many cases ATG triplets were spanning over two different codons, coding for two separate amino acids. Alternate codons or changing the wobble position were used to remove all ATG triplets, while keeping the amino acid composition of the coding region intact. This synthetic fragment was amplified with MB26 and MB28 primers, to create an overlapping overhang with the plasmid backbone at the fragment's 5′ end and also to create an overlapping overhang with the next fragment at its 3′ end. MB28 primer also incorporates T2A self-cleaving peptide to the 3′ end of EGFR left homology arm.

mCherry with membrane localization signal peptide was amplified from pCS-memb-cherry plasmid (Addgene plasmid number: 53750) with MB27 and MB29 primers. EGFR right homology arm was amplified from genomic DNA isolated from NCI-H1975 cell line with MB30 and MB32 primer.

The next DNA fragment inserted into the plasmid contains the PRE-element and overlaps with the EGFR right homology arm at the 5′ end and overlaps with the gRNA scaffold at its 3′end. The EGFR mutation targeting guide RNA was incorporated in this fragment by MB34 primer. This fragment was amplified by primers MB31 and MB34 from pL-CRISPR.EFS.GFP (Addgene plasmid #57818).

The final fragment was amplified by the primer pair MB33 and MB35 from pL-CRISPR.EFS.GFP (Addgene plasmid #57818) plasmid. This fragment overlaps with the single guide RNA sequence at its 5′ end and its 3′end overlaps with the pShuttle plasmid backbone.

Primer MB33 incorporates the mutation specific gRNA sequence. Besides, this fragment also contains gRNA scaffold region, cPPT/CTS region, EF-1alpha core promoter driving Cas9 coding sequence followed by self-cleaving peptide P2A and EGFP (in frame with the Cas9), followed by WPRE element, 3′ LTR and, SV40 poly adenylation signal for proper termination of the Cas9: P2A: EGFP transcript.

pShuttle plasmid was linearized by restriction enzyme at the MCS and the plasmid backbone fragment was separated by agarose gel electrophoresis and purified by Monarch gel extraction kit (NEB) following manufacturer's protocol. All fragments mentioned above (inserts) were amplified by PCR, separated by agarose gel electrophoresis and purified by NEB monarch DNA gel extraction kit.

Purified plasmid backbone and insert fragments were mixed according to manufacturer suggested molar ratio and incubated in a PCR tube with equal volume of 2× NeBuilder master mix at 50° C. in a thermal cycler block for 1 hour. After one hour, 2 μl of the Assembled DNA was mixed with 100 μl NEB10beta electrocompetent cells kept in a BioRad gene pulser cuvette (Bio Rad 165-3083) and electroporation was performed using BioRad Micropulser electroporation system. After electroporation cells were allowed to grow in antibiotic free SOC medium for 1 hour at 37° C. and plated on Kanamycin (30 ug/ml) containing LB agar plates and incubated overnight in an incubator set at 37° C. Positive colonies were identified by colony PCR, replica plated, followed by sequence verification using Sanger sequencing.

Modification Mcherry Cas9 plasmid: inserting 2 BstBI sites: To make the transfer plasmid compatible with the strategy of Miciak J J et. Al. Mcherry Cas9 plasmid was edited to include two BstBI sites, as well as replacing the long homology region of the AdEasy strategy with a 23 bp sequence (ctctactaccttgacctacgaga) that overlaps with pAd5-B2 plasmid, that aids in NeBuilder mediated adenoviral genome assembly following Maiciak J J et. al. One BstBI site was inserted immediately after this overlapping sequence, and another BstBI site was inserted immediately before the ITR-Ad5 Y′ sequence.

Insertion of two BstBI sites was done as a two fragment DNA assembly. From Mcherry Cas9 plasmid, the entire insert was amplified by PCR by primer pair MB74 and MB76 and the 10.5 kb fragment was purified from gel. BstBI sites were integrated in the amplified fragment by these sets of primer. The plasmid backbone was linearized by PCR using primer pair MB75 and MB77, using Mcherry Cas9 plasmid as a template and the resulting 6066 bp DNA fragment was purified from an agarose gel. Insert and the plasmid backbone were assembled together using NeBuilder DNA assembly, and the assembled plasmid was transformed in NEB 10 beta electrocompetent cells by electroporation following the protocol mentioned above. After transformation, cells were plated on Kanamycin plate and positive colonies were identified by colony PCR and Sanger sequencing.

| Primer ID | Sequence (SEQ ID NO: 21-24) |
|---|---|
| MB74 | atcgaattaattcttaattaaTTCGAAcatcatcaataatataccttattttggattgaagc |
| MB75 | caatccaaaataaggtatattattgatgatgTTCGAAttaattaagaattaattcgatcctg |
| MB76 | ccaacggcgttccagacacggTTCGAAtctcgtaggtcaaggtagtagagtttgcgggcagg |
| MB77 | cctgcccgcaaactctactaccttgacctacgagaTTCGAAccgtgtctggaacgccgttgg |

Isolation of plasmid Mcherry Cas9 plasmid and transfection in H1975 and H1650 cells: After verification of the positive clone by sequencing, it was inoculated (from the replica plate) in 100 ml LB with 50 μg/ml Kanamycin, and the plasmid was isolated from overnight culture by alkaline lysis followed by phenol-chloroform-isoamyl alcohol extraction method. The isolated plasmid was further purified by sodium acetate and ethanol precipitation method and finally resuspended in nuclease free water.

Transfection of plasmid (Mcherry Cas9 plasmid) in H1975 and H1650 cells: To deliver plasmid in H1975 and H1650 cells, a 4D-Nucleofector electroporation system (Lonza) was used. Lonza SF cell line 4D nucleofector X kit L (Lonza cat. No. V4XC-2012) was used following manufacturer's protocol to transfect the plasmid in the above mentioned cell lines.

Briefly, H1975 and H1650 cells are cultured following the protocol mentioned before. Before electroporation, cells were resuspended by addition of trypsin-EDTA solution and 0.5×10⁶ cells of each cell line were resuspended in the electroporation buffer provided in the kit and taken in two separate electroporation cuvettes (included in the kit mentioned above). 1 μg plasmid DNA was added to the electroporation cuvette containing resuspended cells. Electroporation was done using the preprogrammed pulsing code EW-127.

After electroporation cells were plated in a glass-bottom 6 well plate in two separate wells. Cells were imaged in an Invitrogen Evos, inverted epi fluorescence microscope using 10× and 20× objective.

### Example II

### Production of Mutation-Specific Replicating Adenovirus in H1975 Cell Line

Delivery of therapeutics in sufficient quantities to tumors can be a major obstacle. To address delivery of therapeutics to tumors, the mutation-specific delivery method described herein is used to engineer mutation-specific replication of adenoviruses. Such a virus replicates specifically in cancer cells, but not in healthy cells, minimizing off-target effects and greatly increasing the viral dose in the tumor. Such viruses encode for a toxin or toxins that would only be expressed in the tumors by mutation-specific delivery strategy outlined above after induction with a drug.

To achieve viral replication dependent on mutations, the same oncogenic EGFR point mutation used for targeted protein delivery was used here. Non-essential genes were deleted from the Ad5 genome to make room for the Cas9 gene delivery construct outlined above. But instead of mcherry, the cargo gene is Iva2, a protein that is essential for packing adenovirus genomes into the viral capsid and that also plays a role for inducing the production of capsid proteins from the viral genome. The Iva2 gene was also deleted in the viral genome at its native locus. Therefore, only cells that carry the correct point mutation and are successfully edited should produce infectious viral particles. As an alternative strategy, a virus was made using the E1A as a cargo gene, which is another protein that is essential for viral replication and can be used for engineering mutation-specific replication.

Indeed, when the virus was tested in H1975 cells with the correct EGFR point mutation, single fluorescent cells grew into clusters of fluorescent cells (FIG. 3A and FIG. 3B). The cluster shown in FIG. 3A and FIG. 3B, contains roughly 70 cells after two weeks in cell culture. In contrast, no viral replication was detected after 2 weeks in H1650 cells, which do not have the targeted EGFR point mutation.

The following describes detailed experimental protocols.

An adenovirus genome was engineered to make an adenovirus that self-replicates specifically in H1975 cells based on a point mutation in the EGFR gene. This strategy involved engineering a minimal genome of adenovirus that lacks one essential protein (Iva2) required for viral replication, as well as removing non-essential genes in E1 and E4 regions to free up space for the construct. The same adenoviral genome encoded Cas9 (driven by EF1α promoter), and a single guide RNA (driven by U6 promoter) that targets a specific point mutation in the EGFR gene in H1975 cells, based on the adjacent PAM site. Cas9 and the delivered guide RNA induces a double stranded break in the EGFR gene. Nucleic acid encoding self-cleaving peptide T2A in frame with Iva2 ORF was also delivered, flanked at its 5′ and 3′ end by two 750 base pair regions from the EGFR gene around the double stranded break. This DNA sequence serves as a template for homology dependent repair after the induction of double stranded break by CRISPR Cas9. Provided homology dependent repair happens in the EGFR gene, T2A:Iva2 ORF gets seamlessly integrated in frame with the EGFR coding sequence. As a result of in-frame seamless integration, truncatedEGFR:T2A:Iva2 fusion protein is made. Due to the presence of T2A peptide in this fusion protein, Iva2 peptide is cleaved out from the fusion protein and released. The construct is designed to stop leaky expression of Iva2 from the viral DNA before integration, which serves as the template for homology dependent repair. Stop codon was added in all 3 frames at the beginning of left homology arm and all ATG triplets were removed from that homology arm. This causes pre-mature termination of the EGFR protein, but production of Iva2 protein ensures self-replication of the adenovirus.

In this strategy, the constructed adenovirus can only replicate in H1975 cells, provided the point mutation present in EGFR gene is targeted by the CRISPR Cas9 and homology dependent repair mediated in-frame integration of T2A:Iva2 ORF takes place.

Strategy for Engineering adenoviral genome: As described earlier, in the adenobuilder strategy described by Miciak J J et al., the adenoviral genome is distributed over 7 plasmids (pAd5, B1 to B7). Each plasmid has two BstBI sites flanking the fragment of adenoviral genome fragment, and digestion with BstBI releases a double stranded fragment from each plasmid and each fragment. Each released double stranded fragment shares an overlap with the successive fragment and 7 fragments can be assembled by NeBuilder DNA assembly in a single reaction. Assembled fragments reconstitute the adenoviral genome as a linear double stranded fragment which can be delivered in packaging cell line like HEK cells. In this strategy, B1 fragment serves as the cargo delivery plasmid and contains a multiple cloning site. The strategy described by Miciak et al. allows easy manipulation of adenoviral genome but each batch of viral preparation requires digestion of constituent plasmids and assembly.

The modularity of the method described by. Miciak J J et al. for modifying other regions of the adenovirus outside the region that can be modified with AdEasy was used. However, the strategy was modified to create a circularized single plasmid, which can be transformed and propagated in NEB10 beta E. coli cells. In the original strategy described by Miciak J J et al., each adenoviral plasmid contains 2 BstBI sites, which releases a double stranded linear fragment on digestion. The strategy was modified to ligate the ends of assembled adenoviral genome to create a circularized single plasmid which can be transformed and propagated in NEB10 beta E. coli cells. To achieve this, the transfer plasmid, which carries the cargo DNA, was reengineered by modifying Mcherry Cas9 plasmid, to contain single BstBI site and, this re-engineered transfer plasmid is constructed on pShuttle backbone and named as workhorse plasmid. Presence of the single BstBI site ensures linearization of the plasmid and then the plasmid backbone can be assembled by NeBuilder DNA assembly with other adenoviral genome fragments, generated by digesting pAd5-B2 to pAd5-B7 plasmids. Large amounts of plasmid DNA can be harvested from NEB10 beta E. coli cells and digestion with PacI separates the adenoviral genome from the plasmid backbone. This fragment is then used to transfect mammalian cells, relying less on replication of a small number of transfected cells as in the original adenobuilder strategy.

In sum, the following steps were carried out.
Step 1: In the workhorse plasmid, mCherry fragment was replaced by adenoviral Iva2 ORF. Also, to allow the virus to self-replicate, the essential Ad5 ELA gene was incorporated in this construct.
Step 2: Assembly of adenoviral genome by NeBuilder, this involves combining Iva2 containing plasmid (in pShuttle backbone) with other modified adenoviral fragments.
Step 3: Isolation of assembled adenoviral DNA, linearization and delivery in H1650 and H1975 cells by electroporation (using Lonza Nucleofector4D).
Step 4: Observation of viral propagation by spinning-disc confocal microscopy.

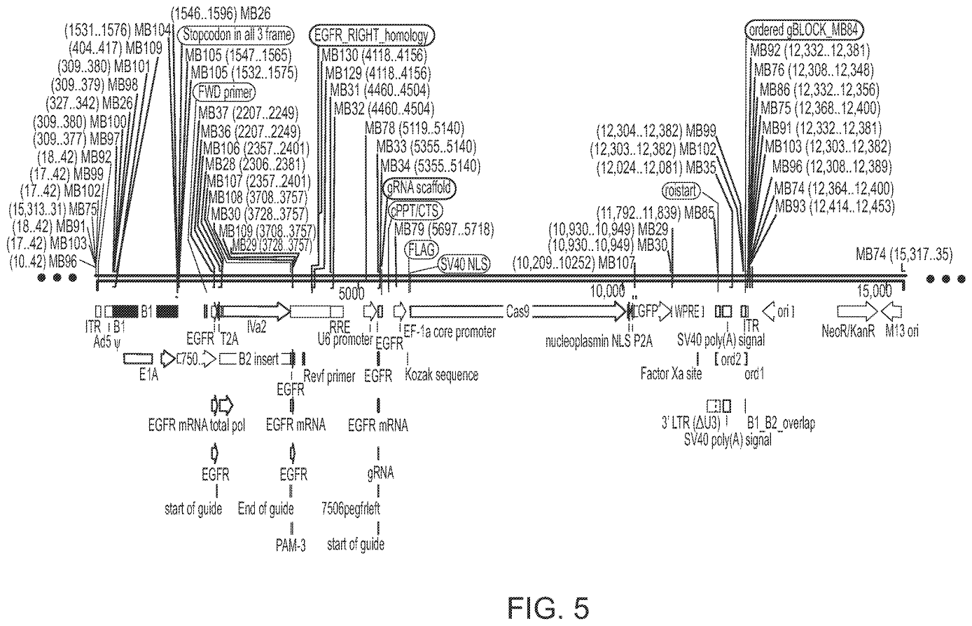

Step 1: Construction of the E1A and Iva2-ORF containing ‘workhorse’ plasmid, the plasmid architecture of which is depicted in FIG. 5. Workhorse plasmid was produced by shortening the length of Mcherry Cas9 plasmid (deleting unnecessary bases from its pShuttle backbone). mCherry Cas9 plasmid was digested by Nsil and SnaBI and the resulting 12.9 kb fragment was separated by agarose gel electrophoresis, excised from gel and purified by Monarch DNA gel extraction kit (NEB). The smaller 3.5 kb fragment was discarded. mCherry Cas9 plasmid contained two BstBI sites, both of which are removed in this digestion step. One single BstBI site was later re-introduced by the cloning strategy below.

Two ends of the digested plasmid were ligated using two double stranded oligo and seamlessly assembled using NeBuilder 2× master mix. The first double stranded fragment was obtained as a gene block from IDT (MB84). This fragment was used as a template for PCR and amplified using primer pair MB85 and MB91 (amplicon size 590 bp). PCR amplification generates overhang with plasmid backbone and also with the second double stranded fragment. The second double stranded fragment was PCR amplified from Mcherry Cas9 plasmid with primer pair MB92 and MB93 (amplicon size 122 bp). This amplified fragment shares overhang with the plasmid backbone and the first double stranded fragment. Both PCR products were separated by agarose gel electrophoresis, bands were excised and purified by Monarch DNA gel extraction kit (NEB). Three above mentioned double stranded fragments (12.9 kb,590 bp, and 122 bp) were seamlessly assembled by NeBuilder 2× master mix to generate the workhorse plasmid.

The assembled plasmid was transformed in NEB10 beta electrocompetent cells following the electroporation protocol mentioned before and plated on LB Kanamycin plate. Positive colonies were identified by colony PCR and further verified by Sanger sequencing. The plasmid was isolated by growing a positive colony in LB overnight.

Downstream modification of workhorse plasmid for mutation-specific adenoviral replication (Iva2 workhorse plasmid) was carried out as follows. Workhorse plasmid was digested by KpnI and Bsu36I. The fragment containing EGFR left homology arm, T2A self-cleaving peptide, mCherry, and part of EGFR right homology arm (˜1.95 kb) was discarded by gel electrophoresis and the rest of the plasmid backbone was excised from the gel and purified by monarch DNA gel purification kit (NEB).

Fragment 1 is described as follows. ELA gene was PCR amplified from pAd5-B1 plasmid (Miciak J J et al.) (Miciak J J, Hirshberg J, Bunz F (2018) Seamless assembly of recombinant adenoviral genomes from high-copy plasmids. PLOS ONE 13 (6): e0199563. doi.org/10.1371/journal.pone.0199563) by MB100 and MB105 primer pair. This fragment at its 5′ terminal overlaps with the plasmid backbone and overlaps with EGFR left homology arm at its 3′ end.

Fragment 2 is described as follows. EGFR left homology arm (stop codon in all 3 frames at its 5′ end and all ATG removed) with T2A self-cleaving peptide at its 3′ end was PCR amplified from previously described mCherry Cas9 plasmid with primer pair MB104 and MB107. This fragment overlaps with fragment 1 at its 5′ end and overlaps with Iva2 ORF at its 3′ end.

Fragment 3 is described as follows. Iva2 ORF was PCR amplified by primer pair MB106 and MB109 from pAd5-B2 plasmid (Miciak J J et al.). This fragment overlaps with fragment 2 at its 5′ end and overlaps with EGFR right homology arm at its 3′ end.

Fragment 4 is described as follows. EGFR right homology arm was PCR amplified by primer pair MB108 and MB129. This fragment overlaps with Iva2 ORF (fragment 3) at the 5′ end and overlaps with the plasmid backbone at Bsu36I site.

All PCR amplifications were done using Q5-2× high fidelity hot start DNA polymerase master mix (NEB, M0494S) and purified by extraction from agarose gel by Monarch DNA gel extraction kit (NEB). Plasmid backbone and fragments mentioned above were assembled and ligated together using NeBuilder 2× HiFi DNA assembly master mix (New England Biolabs, M5520AA) DNA assembly method, following the manufacturer's protocol. The assembled plasmid was transformed into 100 μl NEB10 beta electrocompetent cells following the protocol outlined above. After transformation, cells were plated on LB-kanamycin plate and positive colonies were identified by colony PCR and further confirmed by Sanger sequencing. The resulting plasmid is referred to as Iva2 workhorse plasmid.

Modification of pAd5-B7 plasmid: removal of non-essential adenoviral genes. pAd5-B7 plasmid is one of the constituent plasmids of the adenobuilder adenoviral genome assembly strategy by Miciak J J et al. Non-essential adenoviral genes (E4 ORF1, E4 ORF B, E4 ORF3 and E4 34K) were deleted from this plasmid to shorten the length of overall adenoviral genome and to make room for more cargo DNA. E4 ORF 6/7 is essential in the absence of E4 ORF3 (see, Huang and Hearing, Adenovirus Early Region 4 Encodes Two Gene Products with Redundant Effects in Lytic Infection, JOURNAL OF VIROLOGY, June 1989, p. 2605-2615 DOI: 10.1128/JVI.63.6.2605-2615.1989), and contains an intron, which codes for non-essential gene E4 34K. The intron in E4 ORF 6/7 was deleted and two exons of E4 ORF 6/7 were combined. This modification also removed most of the E4 34K gene.

Fragment 1 is described as follows. Plasmid backbone was linearized by Primer pair MB19 and MB18, and the resulting 5.7 kb DNA fragment was isolated from gel. MB 19 primer is designed to bind at the beginning of the second exon of E4 ORF 6/7.

Fragment 2 is described as follows. E4 ORF 4 and the first exon of E4 ORF 6/7 was amplified by primer pair MB17 and MB20, and the resulting 0.5 kb DNA fragment was purified from gel.

Fragment 1 and fragment 2 were assembled using Nebuilder 2×DNA assembly master mix and the assembled product was transformed into 100 μl NEB 10 beta electro competent cells following the protocol described above and plated on LB ampicillin plate. Positive colonies were selected by colony PCR and confirmed by Sanger sequencing. The resulting plasmid is referred to as pAd5-B7-Del-(E4 ORF1 E4 ORF B E4 ORF3 34K).

| Primer | |
|---|---|
| ID | Sequence (SEQ ID NO: 25-28) |
| MB17 | TTTACTGGTAAGGCTGACTGTTAAAAAAAAaCATGGTTCTTCCAGCTCTTCCC |
| MB18 | CGGGAAGAGCTGGAAGAACCATGtTTTTTTTTAACAGTCAGCCTTACCAGTAA |
| MB19 | GAATGTAACACTTTGACAATGCACAACGCGTGGACTTCTCCTTCGCCGCCCG |
| MB20 | TGCTTAACGGGCGGCGAAGGAGAAGTCCACGCGTTGTGCATTGTCAAAGTG |

Modification of pAd5-B2 plasmid: removal of Iva2 gene. pAd5-B2 plasmid is one of the constituent plasmid of the strategy described by Miciak J J et al. The Iva2 gene was deleted from this plasmid.

Fragment 1 is described as follows. One 802 bp fragment was PCR amplified from pAd5-B2 plasmid by primer pair MB26 and MB53, and purified from gel.

Fragment 2 is described as follows. 807 bp fragment was PCR amplified from pAd5-B2 plasmid with primer pair MB25 and MB54.

Overlap extension PCR using primer MB53 and MB54 was used to join fragment 1 and fragment 2, creating 1.6 kb DNA fragment 1-2, which was purified from agarose gel.

Fragment 3 (plasmid backbone) is described as follows. pAd5-B2 plasmid was digested by Clal and SphI, and the 8.7 kb plasmid backbone fragment was purified from gel.

Fragment 1-2, and 3 were assembled by Nebuilder DNA assembly master mix, and the assembled plasmid was transformed in NEB 10 beta electro competent cells following the protocol mentioned before and plated on LB ampicillin plates. Positive colonies were selected by colony PCR and confirmed by Sanger sequencing. The resulting plasmid is referred to as pAd5-B2-Del-Iva2.

| Primer ID | Sequence (SEQ ID NO: 29-32) |
|---|---|
| MB25 | GCTGGATCGAGATGCCGTAGAGACAGCAAGACACTTGCTTGATCCAAATCCAAACAG |
| MB26 | TGGATTTGGATCAAGCAAGTGTCTTGCTGTCTCTACGGCATCTCGATCCAGCATATC |
| MB53 | CCTTCCTTGATGCCGAAGAGGGCGACATGTGTCTTCACACCCTGGAGCGAGTGGACCC |
| MB54 | tatgagcggataataatttcacacaggaggtttaaactttaaacatgtcaaaagagacg |

Step 2: Adenoviral plasmid assembly by NeBuilder DNA assembly method. Iva2 workhorse plasmid was linearized by BstBI and the resulting plasmid works as the plasmid backbone for adenoviral plasmid assembly. Plasmids pAd5-Del-Iva2, pAd5-B3, pAd5-B4, pAd5-B5, pAd5-B6 Del-E3 and pAd5-B7-Del-(E4 ORF1 E4 ORF B E4 ORF3 34K) were digested by BstBI. Each of these plasmids contain two BstBI sites, flanking the adenoviral genomic elements. On BstBI digestion, adenoviral genomic elements were released as double stranded DNA fragments and purified from gel. Linearized workhorse plasmid and double stranded fragments isolated from pAd5-Del-Iva2, pAd5-B3, pAd5-B4, pAd5-B5, pAd5-B6 Del-E3 and pAd5-B7-Del-(E4 ORF1 E4 ORF B E4 ORF3 34K) were assembled together by NeBuilder DNA assembly method. The assembled plasmid was transformed into 100 μl NEB10 beta electrocompetent cells following the protocol mentioned before. After transformation cells were plated on LB-kanamycin plate and positive colonies were identified by colony PCR and further confirmed by restriction digestion.

Step 3: Isolation of assembled adenoviral plasmid and delivery in H1975 and H1650 cells. Assembled adenoviral plasmid was isolated from overnight LB culture (containing Kanamycin 50 μg/ml) by alkaline lysis, followed by phenol chloroform extraction and ethanol precipitation. Isolated plasmid was digested with PacI, and the ˜38 kb fragment, which represents the assembled adenoviral genome, was purified from gel. Purified DNA was re-precipitated by ethanol and sodium acetate and the resuspended in de-ionized water.

1 μg of linearized DNA was electroporated into 0.5×10⁶ H1975 and H1650 cells respectively using the Lonza SF cell line 4D nucleofector X kit L (Lonza cat. No. V4XC-2012), following the protocol described above. After electroporation, cells were plated in a glass-bottom 6 well plate in two separate wells.

Step 4: Observation of viral propagation by Spinning-Disc confocal microscopy. Cells were imaged in a custom-built Spinning-Disc (Yokogawa) inverted confocal microscope, using 10× and 20× objective.

| Oligo | |
|---|---|
| ID | Sequence (SEQ ID NO: 33-53) |
| MB26 | ctcatagcgcgtaatactggtaccgcTAATAGTGACTAATAGTGACTAATAGTGAtctcacatattattcctttc |
| MB27 | catgtcaagatcacagattttgagggcagaggaagtctgctaacatgcggtgacgtcgaggagaatcctggacctgtgagca |
| | agggcgaggagg |
| MB28 | cctcctcgcccttgctcacaggtccaggattctcctcgacgtcaccgcatgttagcagacttcctctgccctcaaaatctgt |
| | gatcttgacatg |
| MB29 | ctcttccgcacccagcagtttggccCgccccttgtacagctcgtccatgc |
| MB30 | gcatggacgagctgtacaaggggcGggccaaactgctgggtgcggaagag |
| MB31 | cacagagggtgctcagaaaggagctttgttccttgggttcttggg |
| MB33 | ggacgaaacaccGTCAAGATCACAGATTTTGGGgttttagagctagaaatagcaag |
| MB34 | cttgctatttctagctctaaaacCCCAAAATCTGTGATCTTGACggtgtttcgtcc |
| MB35 | cgatatctctagactcgaggctaagatacattgatgagtttggacaaaccacaactag |
| MB84 | Gene Block |
| MB85 | cgtctgttgtgtgactctggtaactagagatccctcagacccttttag |
| MB91 | cttattttggattgaagccaatatggtttaaacTTCGAAtctcgtaggtc |
| MB92 | gacctacgagaTTCGAAgtttaaaccatattggcttcaatccaaaataag |
| MB93 | cttacgcatctgtgcggtatttcacaccgcatatggatcc |
| MB100 | ctgaataattttgtgttactcatagcgcgtaatATTTGTCTAGGGCCGCGGGGACTTTGACCGTTTACGTGG |
| MB104 | CGCCCCAGGCCATAAgcTAATAGTGACTAATAGTGACTAATAGTGA |
| MB105 | CACTATTAgTCACTATTAgTCACTATTAgcTTATGGCCTGGGGC |
| MB106 | gacgtcgaggagaatcctggacctGAAACCAGAGGGCGAAGACCG |
| MB107 | CGGTCTTCGCCCTCTGGTTTCaggtccaggattctcctcgacgtc |
| MB108 | CGCGCAAAACCCCTAAATAAgggcGggccaaactgctgggtgcggaagag |
| MB109 | ctcttccgcacccagcagtttggccCgcccTTATTTAGGGGTTTTGCGCG |
| MB129 | ggggcctttcctgttccttaggtgtccttgacagcagtg |

The following additional methods are described.

Preparation of adenovirus by AdEasy method. AdEasy method of adenovirus production was described by He T C et al. (Proc Natl Acad Sci USA. 1998 Mar. 3; 95 (5): 2509-14). This technique involves, recombination of a transfer plasmid and another plasmid (pAdEasy 1 or pAdEasy 2) containing the rest of adenoviral genome, in BJ5183 E. coli cells.

AdEasier2 BJ5183 E. coli cells. AdEasier2 plasmid was transformed in BJ5183 E. coli cells obtained from Addgene and made electrocompetent following the protocol mentioned by He T C et al. (Proc Natl Acad Sci USA. 1998 Mar. 3; 95 (5): 2509-14). Briefly, cells were grown to 0.5 OD590 in LB supplemented with Ampicillin (100 μg/ml). Cells were harvested by centrifugation at 4° C. and washed twice with ice-cold 10% glycerol. After final wash, 100 μl aliquots of cells were made and stored in −80° C. freezer.

Mcherry Cas9 plasmid was linearized with Pmel and electroporated into Adeasier-2 cells. After electroporation, cells were plated on 50 μg/ml kanamycin containing LB-Agar plate and incubated overnight at 37° C. Recombinant colonies are kanamycin resistant and usually small in size. Recombinant colonies were identified by colony PCR and replica plated. Plasmid was isolated from recombinant colonies. Recombinant plasmids were further confirmed by restriction enzyme digestion and agarose gel electrophoresis. After confirmation, recombinant adenoviral plasmid was transformed in Neb10 beta electrocompetent cells and plated on LB Kanamycin (50 μg/ml) plate and incubated overnight at 37° C. Single colonies from this plate were used to inoculate 100 ml LB cultures with 50 μg/ml Kanamycin for large scale plasmid isolation.

Isolated plasmid were linearized and the backbone isolated from the adenoviral genome using PacI digestion. Digested plasmid was purified by phenol-chloroform extraction followed by ethanol and sodium acetate precipitation. Digested plasmid was resuspended in de-ionized water and 1 μg of purified plasmid was taken for electroporation in 911E4 mammalian packaging cell line, following the protocol used by He T C et al.

Modification of mcherry Cas9 plasmid for mutation-specific integration-dependent expression of melittin (major component of honeybee toxin).

Fragment 1 is described as follows. From mcherry Cas9 plasmid, EGFR left homology arm and the in frame T2A peptide was amplified by MB26 and MB150 primer pair. The resulting 879 bp fragment was purified from the gel. This fragment at its 3′ end shares overlap with fragment 2.

Fragment 2 is described as follows. Melittin DNA sequence was synthesized as a gene block. From this gene block, melittin sequence was amplified using MB149 and MB152. In the amplified fragment, elittin lacks any ATG codon at its 5′ end and on assembly it gets in frame with EGFR. The resulting 241 bp fragment was isolated from gel. Fragment 2 shares overlap with fragment 3 at its 3′ end.

Fragment 3 is described as follows. EGFR right homology arm was amplified using primer pair MB151 and MB32, and the resulting 799 bp fragment was purified from gel. This fragment at its 5′ end overlaps with fragment 2 and at 3′ end overlaps with the plasmid backbone.

Preparation of plasmid backbone. mcherry Cas9 plasmid was digested by KpnI and Bsu36I and 11.5 kb plasmid backbone was isolated from gel.

Purified plasmid backbone and insert fragments were assembled together by NeBuilder DNA assembly method and the assembled plasmid was transformed in NEB 10 beta electrocompetent cells by electroporation protocol mentioned before. After transformation, cells were plated on Kanamycin-LB plate and positive colonies were identified by colony PCR, and Sanger sequencing. The resulting plasmid is called melittin Cas9 plasmid. The plasmid can be used together with the AdEasy strategy and AdEasier-2 to move the construct into a non-replicating adenovirus vector for delivery to mammalian cells or animals.

| Primer | |
|---|---|
| ID | Sequence (SEQ ID NO: 54-59) |
| MB26 | ctcatagcgcgtaatactggtaccgcTAATAGTGACTAATAGTGAcTAATAGTGAtctcacatattattcctttc |
| MB149 | gaggagaatcctggacctAAGTTCCTGGTGAACGTGGC |
| MB150 | GCCACGTTCACCAGGAACTTaggtccaggattctcctc |
| MB151 | CAAGAGGAAGAGGCAGCAGGGCgggGggccaaactgc |
| MB152 | gcagtttggccCgcccGCCCTGCTGCCTCTTCCTCTTG |
| MB32 | cccaagaacccaaggaacaaagctcctttctgagcaccctctgtg |

### Example III

### Mutation-Specific Propogation of Engineered Ad5 Adenovirus

Ad5 adenovirus genome was engineered to make it replication deficient by deletion of essential gene IVA2, which packs viral DNA into the viral capsid. Then, the IVA2 ORF (open reading frame, which upon transcription, can code for a protein), without a promoter, start codon or Kozak sequence was incorporated in the adenoviral genome as cargo DNA flanked with DNA sequence from human EGFR gene at its 5′ and 3′ end. Elimination of any promoter sequence, start codon or Kozak sequence at the 5′ end of IVA2 ORF and incorporation of stop codon in all 3 reading frames ensures no protein expression from this piece of DNA before integration into the target cells. The engineered adenoviral genome also carries Cas9 and a guide RNA sequence, specific to a point mutation in human EGFR gene which is only present in NCI H-1975 cells, but not in NCI H-1650 cells. Upon delivery of this engineered adenoviral genome in respective cell lines (H-1650 and H-1975), the IVA2 gene gets integrated in-frame into the EGFR gene only in 1975 cells via homology-dependent repair (HDR), as Cas9 and expression of a mutation-specific guide RNA leads to a double strand DNA break only on H-1975 cells. HDR ensures in frame integration of IVA2 and IVA2 mRNA is transcribed under endogenous EGFR promoter. According to one aspect, the above method can target other mutations like protein fusions, DNA translocations or frameshift mutations, as described with respect to FIG. 1. FIG. 9 is a schematic diagram of the core element of the cargo delivered through adenoviral genome that enables mutation specific replication of the virus upon in-frame integration in the EGFR gene.

More specifically, engineered adenoviral DNA was delivered in cultured NCI H-1975 cells and NCI H-1650 cells and cells were cultured in RPMI-1650 media (supplemented with 10% FBS and 1× antibiotic-antimycotic cocktail) for long term in a 37° C. humidified incubator. Cells were plated on glass bottom plates. Cells were imaged using a spinning disc confocal microscope. The entire experiment was independently performed twice, several months apart. In both cases, delivery of the Ad5 construct DNA to 1975 cells resulted in replicating virus, as indicated by a growing number of viral clusters that increased in cell number and size over time. In contrast, no replication was observed in 1650 cells in either experiment. Initially transfected 1650 cells expressing Cas9 GFP disappeared over time and no new clusters or growth of clusters were observed.

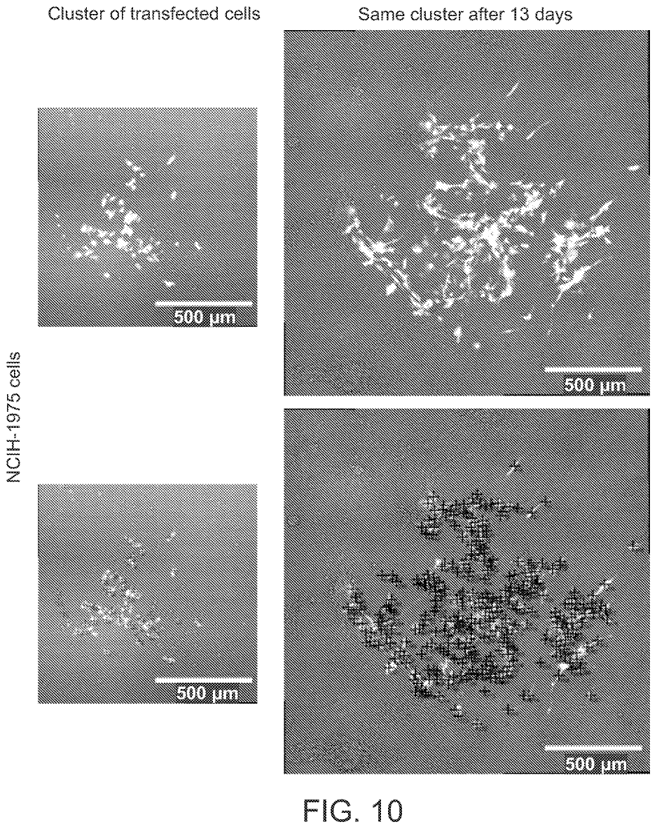

FIG. 10 depicts propagation of mutation-specific replicating adenovirus in NCI H-1975 cells. The upper left image depicts an example cluster of Cas9 GFP expressing cells, indicating the delivery and expression from the viral genome in NCI H-1975 from the second experiment in Example III. The lower left image depicts counted cells in the cluster of the upper left image on day 1. The upper right image is the same cluster of the upper left image 13 days later. Adenovirus replicates and propagates in NCI H-1975 cells, as the original cell cluster progressively increased in size and cell number. The lower right image depicts counted cells in the cluster from the upper right image.

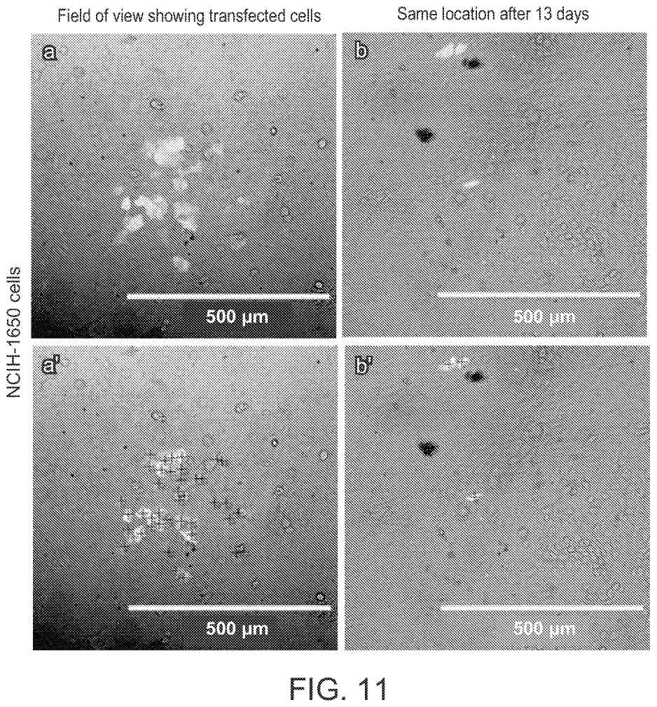

FIG. 11 depicts that mutation-specific replicating adenovirus did not replicate in NCI H-1650 cells that lack the target mutation. Upper left image a is a field of view of transfected cells, expressing Cas9 GFP in NCI H-1650 cells from the second experiment in Example III. The lower left image a′ depicts segmented cells in the cluster of the upper left image a. Upper right image b depicts the same cluster of upper left image a 13 days later. Adenovirus failed to replicate and propagate in NCI H-1650 cells, as the original cells expressing the Cas9 GFP were progressively lost over time and no new cells or clusters appeared. The lower right image b′ depicts counted cells in the cluster from upper right image b.

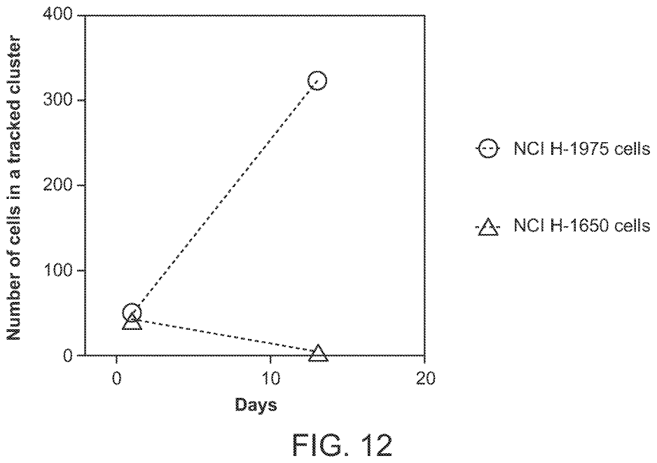

FIG. 12 is a graph of quantification of infected cells expressing Cas9 GFP from FIG. 10 and FIG. 11. The number of Cas9 GFP-expressing cells in H-1975 cells strongly increased over time, whereas the number of Cas9 GFP-expressing cells progressively decreased over time in H-1650 cells.

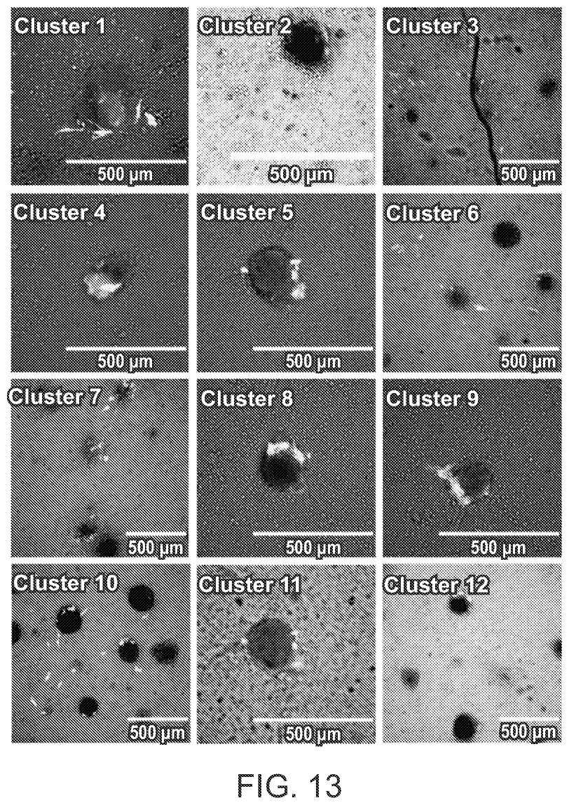

FIG. 13 depict confocal projections of viral clusters found in NCI H-1975 cells about 1.5 months into the first experiment of Example III.

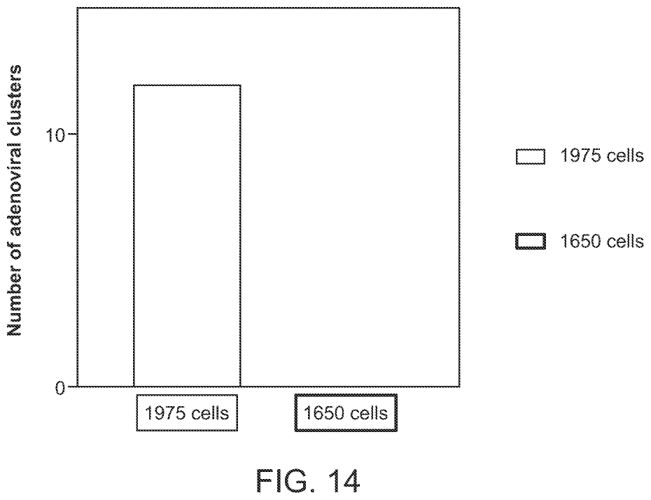

FIG. 14 is a graph of quantification of the total number of observed viral clusters in NCI H-1650 and NCI H-1975 cells about 1.5 months into the first experiment of Example III. In H-1975 cells, 12 replicating viral clusters were found that continued to grow over time. No remaining Cas9 GFP expressing cells were found in H-1650 cells.

According to the data presented above, the methods described herein were used to successfully engineer a version of adenovirus which can replicate in mammalian cells in a mutation specific manner. According to one aspect, the engineered adenovirus does not replicate without in frame integration in the mammalian cell genome. According to one aspect, a stop codon was incorporated in all three coding frames at the 5′ end of the homologous region. According to one aspect, although a PAM point mutation was used in this Example III to target H-1975 cells, the present disclosure contemplates that any cell type can be targeted which has a mutation unique to the cell type resulting in a PAM sequence that can be targeted by a Cas enzyme, such as a Cas enzyme of a Type II CRISPR system, of which Cas9 is an example. As described herein, other mutations resulting in fusion proteins, chromosome translocations, deletions, or duplication of chromosome fragments can be targeted even if the mutation does not result in a PAM site, because adjacent PAM sites for available Cas9 enzymes can typically be found such that the guide RNA can be designed to overlap the chromosome break. Alternatively, other gene editors can be uniquely targeted to the chromosome breakpoint ensuring mutation-specificity. Finally, these types of mutations result in distinct homology arms ensuring mutation-specific integration in the target cells. It is to be understood that the methods described herein can use any sequence unique to a cell intended to be targeted for treatment, such as by insertion of a donor sequence encoding a treatment molecule to be expressed by the cell.

### Example IV

### Embodiments

The present disclosure provides a method of treating a collection of cells including (1) a target mutated cell including a first mutation in cellular DNA and (2) a plurality of cells lacking the first mutation. The method includes the steps of (a) administering to the collection of cells (1) a nucleic acid construct including (i) a first nucleic acid encoding a treatment molecule wherein the nucleic acid construct optionally lacks one or more expression elements, such as for stable expression before integration of the construct and optionally targets the first mutation, and (ii) optionally a second nucleic acid encoding a gene editor, wherein the gene editor is expressed, wherein the gene editor optionally targets the first mutation and cleaves the cellular DNA at a target integration site, wherein at least either the nucleic acid construct or the gene editor targets the first mutation, (b) integrating the first nucleic acid encoding the treatment molecule into the target integration site of the cellular DNA of the target mutated cell, and (c) expressing the first nucleic acid encoding the treatment molecule after integration, wherein the treatment molecule treats the target mutated cell. According to one aspect, the first nucleic acid encoding the treatment molecule is integrated spontaneously into the cellular DNA. According to one aspect, the gene editor cuts or nicks the cellular DNA and the first nucleic acid encoding the treatment molecule is integrated into the cellular DNA. According to one aspect, the collection of cells is homogenous. According to one aspect, the collection of cells is heterogeneous. According to one aspect, a plurality of mutations in a cell are targeted. According to one aspect, a plurality of a nucleic acid constructs comprising a first nucleic acid encoding a treatment molecule are introduced into the cell targeting a plurality of mutations, wherein the treatment molecules of the plurality are the same or different. According to one aspect, the cellular DNA is genomic DNA, mitochondrial DNA, plasmid DNA, exogenous DNA, foreign DNA or viral DNA. According to one aspect, the expression in step (c) is induced by an inducible gene expression system or by removing an expression inhibitor or degradation signal. According to one aspect, the expression inhibitor or degradation signal is removed by an inducible DNA flippase that can remove a transcription or translation terminator or a degradation tag or localization tag flanked by FRT sites, or a stop codon before the first nucleic acid encoding the treatment molecule that inhibits expression. According to one aspect, the target mutated cell is a member selected from the group consisting of a neoplastic cell, a cancer cell, an immune cell, a virus-infected cell, and a pathogen-infected cell. According to one aspect, the target mutated cell is a bacterial cell, an insect cell, a plant cell or an animal cell. According to one aspect, the cellular DNA is a gene. According to one aspect, the plurality of cells comprises wild type cellular DNA lacking the first mutation. According to one aspect, the nucleic acid construct is within a vector. According to one aspect, the vector is a member selected from the group consisting of a virus, a liposome, a microorganism, and a nanoparticle. According to one aspect, the vector lacks a gene expression element for expressing the first nucleic acid. According to one aspect, the nucleic acid construct is within a vector lacking a gene expression element for expressing the first nucleic acid. According to one aspect, the nucleic acid construct is within a vector lacking one or more of a start codon, a ribosome binding site, a promoter, a splicing sequence, a polyA signal, a mRNA processing signal, a transcriptional regulatory sequence, a post-transcriptional regulatory sequence, or a post-translation regulatory sequence. According to one aspect, the nucleic acid construct lacks a gene expression element. According to one aspect, the nucleic acid construct lacks one or more of a start codon, a ribosome binding site, a promoter, a splicing sequence, a polyA signal, a mRNA processing signal, a transcriptional regulatory sequence, a post-transcriptional regulatory sequence, or a post-translation regulatory sequence. According to one aspect, the nucleic acid construct contains a degradation signal of the treatment molecule that is lost after integration into cellular DNA. According to one aspect, the nucleic acid construct includes an expression inhibitor that if removed induces expression of the first nucleic acid. According to one aspect, the expression inhibitor is a stop codon, terminal signal, transcription terminator or translation terminator. According to one aspect, the nucleic acid construct includes a 5′ flanking nucleic acid and a 3′ flanking nucleic acid, wherein the 5′ flanking nucleic acid and the 3′ flanking nucleic acid are homologous to the target integration site, and wherein the 5′ flanking nucleic acid lacks a gene expression element. According to one aspect, the nucleic acid construct includes a 5′ flanking nucleic acid and a 3′ flanking nucleic acid, wherein the 5′ flanking nucleic acid is homologous to the first mutation in the genomic DNA associated with the target mutated cell and the 3′ flanking nucleic acid is homologous to wild type genomic DNA, and wherein the 5′ flanking nucleic acid lacks a start codon. According to one aspect, the optional gene editor is a member selected from the group consisting of a CRISPR system, a TALEN, a zinc finger nuclease or a restriction enzyme. According to one aspect, the first mutation is a mutation associated with a cancer cell or an immune cell. According to one aspect, the first mutation is a point mutation, a frameshift mutation, a translocation, an inversion, an insertion, a deletion, a duplication, a nucleic acid encoding a protein fusion or a viral DNA integration. According to one aspect, the gene editor is a CRISPR system comprising a Cas enzyme and a guide RNA, wherein a PAM sequence recognized by the Cas enzyme for nuclease activity includes the first mutation. According to one aspect, the first mutation is a point mutation, the gene editor is a CRISPR system comprising a Cas enzyme and a guide RNA, wherein the targeted mutation is located in the binding region of the guide RNA adjacent a PAM sequence recognized by the Cas enzyme for nuclease activity. According to one aspect, the treatment molecule is a toxin that is expressed by the target mutated cell. According to one aspect, the toxin is an AB toxin. According to one aspect, the toxin is a member selected from the group consisting of ricin, Shiga toxin 1 and 2, Cholera toxin, Melittin, Phospholipases A and C, Streptolysin O and S, Pertussigen, Clostridium difficile TcdB, Sphingomyelinase C, Staphylococcus aureus alpha toxin, Staphylococcus aureus beta toxin, and Staphylococcus aureus delta toxin. According to one aspect, the treatment molecule is a toxin that is expressed by the target mutated cell resulting in cell death of the target mutated cell. According to one aspect, the treatment molecule is a toxin that is expressed by the target mutated cell resulting in cell death of the target mutated cell and surrounding cells. According to one aspect, the treatment molecule is an enzyme that convert a cellular metabolite into a toxic compound. According to one aspect, the enzyme is HCN synthase hcnABC from Pseudomonas and produces cyanide from glycine. According to one aspect, the treatment molecule is a member selected from the group consisting of an RNA, an immune antigen, an immunomodulatory factor, a nanobody, a modulator of a signal transduction pathway, an enzyme or enzyme subunit, a dominant negative form of a cell signaling pathway intermediate, and a transcription factor, and the like. According to one aspect, the gene editor targets the first mutation. According to one aspect, the first nucleic acid targets the first mutation. According to one aspect, the gene editor and the first nucleic acid target the first mutation. According to one aspect, the method further includes repressing nonhomologous end joining in the target mutated cell by expression of peptides or RNA interference. According to one aspect, the method further includes a nucleic acid encoding an in-frame self-cleaving peptide. According to one aspect, the first nucleic acid and the second nucleic acid are within a vector delivered to the target cell. According to one aspect, the vector is a virus vector. According to one aspect, the virus vector is an adenovirus vector. According to one aspect, the adenovirus vector is Ad5. According to one aspect, the virus vector replicates within the target mutated cell. According to one aspect, the virus vector excludes a gene required for or enhances replication and includes a third nucleic acid encoding the gene required for or enhances replication, wherein the third nucleic acid is inserted into the cellular DNA of the target mutated cell, wherein the third nucleic acid encoding the gene required for or enhancing replication is expressed, and the virus replicates within the target mutated cell. According to one aspect, the gene required for replication is Iva2 or E1A. According to one aspect, the first mutation is a viral DNA integration resulting in a unique DNA sequence or a PAM site in the mutated cell.

The present disclosure provides a system for targeting mutations in cancer cells including a first nucleic acid encoding a treatment molecule wherein the first nucleic acid optionally targets the first mutation, and a second nucleic acid encoding a gene editor, wherein the gene editor optionally targets the first mutation, and wherein at least either the first nucleic acid or the gene editor targets the first mutation. According to one aspect, the system further includes a third nucleic acid encoding a gene required for or enhancing viral replication.

The present disclosure provides a viral vector including a system for targeting mutations in cancer cells including a first nucleic acid encoding a treatment molecule wherein the first nucleic acid optionally targets the first mutation, and a second nucleic acid encoding a gene editor, wherein the gene editor optionally targets the first mutation, and wherein at least either the first nucleic acid or the gene editor targets the first mutation.

The present disclosure provides a viral vector including a system for targeting mutations in cancer cells including a first nucleic acid encoding a treatment molecule wherein the first nucleic acid optionally targets the first mutation, and a second nucleic acid encoding a gene editor, wherein the gene editor optionally targets the first mutation, and wherein at least either the first nucleic acid or the gene editor targets the first mutation, wherein the system further includes a third nucleic acid encoding a gene required for or enhancing viral replication

The present disclosure provides a system for targeting specific DNA sequences such as V(D)J recombination sequences associated with specific immune cells such as B cell or T cells, the system includes a first nucleic acid encoding a treatment molecule wherein the first nucleic acid optionally targets the specific DNA sequence, and a second nucleic acid encoding a gene editor, wherein the gene editor optionally targets the specific DNA sequence, and wherein at least either the first nucleic acid or the gene editor targets the specific DNA sequence.

### Other Embodiments

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

1. A method of treating a collection of cells comprising (1) a target mutated cell including a first mutation in cellular DNA and (2) a plurality of cells lacking the first mutation comprising
(a) administering to the collection of cells (1) a nucleic acid construct comprising
(i) a first nucleic acid encoding a treatment molecule wherein the nucleic acid construct optionally lacks one or more expression elements, such as for stable expression before integration of the construct and optionally targets the first mutation, and
(ii) optionally a second nucleic acid encoding a gene editor, wherein the gene editor is expressed, wherein the gene editor optionally targets the first mutation and cleaves the cellular DNA at a target integration site,
wherein at least either the nucleic acid construct or the gene editor targets the first mutation,
(b) integrating the first nucleic acid encoding the treatment molecule into the target integration site of the cellular DNA of the target mutated cell, and
(c) expressing the first nucleic acid encoding the treatment molecule after integration, wherein the treatment molecule treats the target mutated cell.

2. The method of claim 1 wherein the first nucleic acid encoding the treatment molecule is integrated spontaneously into the cellular DNA or wherein the gene editor cuts or nicks the cellular DNA and the first nucleic acid encoding the treatment molecule is integrated into the cellular DNA.

3.-5. (canceled)

6. The method of claim 1 wherein a plurality of mutations in a cell are targeted.

7. (canceled)

8. The method of claim 1 wherein the cellular DNA is genomic DNA, mitochondrial DNA, plasmid DNA, exogenous DNA, foreign DNA or viral DNA.

9. The method of claim 1 wherein the expression in step (c) is induced by an inducible gene expression system or by removing an expression inhibitor or degradation signal.

10. (canceled)

11. The method of claim 1 wherein the target mutated cell comprises a neoplastic cell, a cancer cell, an immune cell, a virus-infected cell, or a pathogen-infected cell, or wherein the target mutated cell comprises a bacterial cell, an insect cell, a plant cell or an animal cell.

12.-13. (canceled)

14. The method of claim 1 wherein the plurality of cells comprises wild type cellular DNA lacking the first mutation.

15.-19. (canceled)

20. The method of claim 1 wherein the nucleic acid construct lacks a gene expression element, or wherein the nucleic acid construct lacks one or more of a start codon, a ribosome binding site, a promoter, a splicing sequence, a polyA signal, a mRNA processing signal, a transcriptional regulatory sequence, a post-transcriptional regulatory sequence, or a post-translation regulatory sequence.

21. (canceled)

22. The method of claim 1 wherein the nucleic acid construct contains a degradation signal of the treatment molecule that is lost after integration into cellular DNA or wherein the nucleic acid construct includes an expression inhibitor that if removed induces expression of the first nucleic acid.

23.-24. (canceled)

25. The method of claim 1 wherein the nucleic acid construct includes a 5′ flanking nucleic acid and a 3′ flanking nucleic acid, wherein the 5′ flanking nucleic acid and the 3′ flanking nucleic acid are homologous to the target integration site, and wherein the 5′ flanking nucleic acid lacks a gene expression element.

26. (canceled)

27. The method of claim 1 wherein the optional gene editor comprises a CRISPR system, a TALEN, a zinc finger nuclease or a restriction enzyme.

28. The method of claim 1 wherein the first mutation is a mutation associated with a cancer cell or an immune cell.

29. The method of claim 1 wherein the first mutation is a point mutation, a frameshift mutation, a translocation, an inversion, an insertion, a deletion, a duplication, a nucleic acid encoding a protein fusion or a viral DNA integration or wherein the first mutation is a viral DNA integration resulting in a unique DNA sequence or a PAM site in the mutated cell.

30.-31. (canceled)

32. The method of claim 1 wherein the treatment molecule is a toxin that is expressed by the target mutated cell, and comprises an AB toxin, ricin, Shiga toxin 1 and 2, Cholera toxin, Melittin, Phospholipases A and C, Streptolysin O and S, Pertussigen, Clostridium difficile TedB, Sphingomyelinase C, Staphylococcus aureus alpha toxin, Staphylococcus aureus beta toxin, or Staphylococcus aureus delta toxin.

33.-34. (canceled)

35. The method of claim 1 wherein the treatment molecule comprises a toxin that is expressed by the target mutated cell resulting in cell death of the target mutated cell, and optionally surrounding cells, or wherein the treatment molecule comprises an enzyme that convert a cellular metabolite into a toxic compound, or wherein the treatment molecule comprises an RNA, an immune antigen, an immunomodulatory factor, a nanobody, a modulator of a signal transduction pathway, an enzyme or enzyme subunit, a dominant negative form of a cell signaling pathway intermediate, or a transcription factor.

36.-41. (canceled)

42. The method of claim 1 wherein the gene editor or the first nucleic acid target the first mutation.

43. The method of claim 1 further comprising repressing nonhomologous end joining in the target mutated cell by expression of peptides or RNA interference.

44. The method of claim 1 further comprising a nucleic acid encoding an in-frame self-cleaving peptide.

45.-52. (canceled)

53. A system for targeting mutations in cancer cells comprising
a first nucleic acid encoding a treatment molecule wherein the first nucleic acid optionally targets the first mutation, and a second nucleic acid encoding a gene editor,
wherein the gene editor optionally targets the first mutation, and wherein at least either the first nucleic acid or the gene editor targets the first mutation.

54. (canceled)

55. A viral vector comprising the system of claim 53.

56.-57. (canceled)


SEQUENCE LISTING (59 sequences)

SEQ ID NO: 1 - DNA, 48 bp, synthetic construct
  misc_feature 1..48: note = EFGR mutation normal
  source 1..48: mol_type = other DNA
aattaagagaagcaacatctccgaaagccaacaaggaaatcctcgatg

SEQ ID NO: 2 - DNA, 36 bp, synthetic construct
  misc_feature 1..36: note = EFGR Mutation deletion
  source 1..36: mol_type = other DNA
aattaagagaagcaacatctcaggaaatcctcgatg

SEQ ID NO: 3 - DNA, 47 bp, synthetic construct
  misc_feature 1..47: note = HRAS mutation normal
  source 1..47: mol_type = other DNA
tggacatcctggataccgccggccaggaggagtacagcgccatgcgg

SEQ ID NO: 4 - DNA, 47 bp, synthetic construct
  misc_feature 1..47: note = HRAS mutation Q61L
  source 1..47: mol_type = other DNA
tggacatcctggataccgccggcctggaggagtacagcgccatgcgg

SEQ ID NO: 5 - DNA, 48 bp, synthetic construct
  misc_feature 1..48: note = BRAF mutation normal
  source 1..48: mol_type = other DNA
taggtgattttggtctagctacagtgaaatctcgatggagtgggtccc

SEQ ID NO: 6 - DNA, 48 bp, synthetic construct
  misc_feature 1..48: note = BRAF mutation V600G
  source 1..48: mol_type = other DNA
taggtgattttggtctagctacagggaaatctcgatggagtgggtccc

SEQ ID NO: 7 - DNA, 48 bp, synthetic construct
  misc_feature 1..48: note = BRAF mutation V600E
  source 1..48: mol_type = other DNA
taggtgattttggtctagctacagagaaatctcgatggagtgggtccc

SEQ ID NO: 8 - DNA, 20 bp, synthetic construct
  misc_feature 1..20: note = mutation
  source 1..20: mol_type = other DNA
gtgattttggtctagctaca

SEQ ID NO: 9 - DNA, 20 bp, synthetic construct
  misc_feature 1..20: note = mutation
  source 1..20: mol_type = other DNA
cctggataccgccggcctgg

SEQ ID NO: 10 - DNA, 20 bp, synthetic construct
  misc_feature 1..20: note = mutation
  source 1..20: mol_type = other DNA
ggcgctgtactcctccaggc

SEQ ID NO: 11 - DNA, 20 bp, synthetic construct
  misc_feature 1..20: note = mutation
  source 1..20: mol_type = other DNA
tctctctgaaatcactaagc

SEQ ID NO: 12 - DNA, 20 bp, synthetic construct
  misc_feature 1..20: note = mutation
  source 1..20: mol_type = other DNA
caaatgaatgatgcacgtca

SEQ ID NO: 13 - DNA, 20 bp, synthetic construct
  misc_feature 1..20: note = mutation
  source 1..20: mol_type = other DNA
atgaatgatgcacgtcatgg

SEQ ID NO: 14 - protein, 7 aa, synthetic construct
  REGION 1..7: note = spacer sequence
NNAGAAW

SEQ ID NO: 15 - protein, 4 aa, synthetic construct
  REGION 1..4: note = protospacer
NAAR

SEQ ID NO: 16 - protein, 4 aa, synthetic construct
  REGION 1..4: note = PAM
TTTV

SEQ ID NO: 17 - protein, 4 aa, synthetic construct
  REGION 1..4: note = PAM
TYCV

SEQ ID NO: 18 - protein, 4 aa, synthetic construct
  REGION 1..4: note = PAM
TATV

SEQ ID NO: 19 - DNA, 25 bp, synthetic construct
  misc_feature 1..25: note = EGFR gene region of interest
  source 1..25: mol_type = other DNA
gtcaagatcacagattttgggcggg

SEQ ID NO: 20 - DNA, 25 bp, synthetic construct
  misc_feature 1..25: note = EGFR gene region of interest
  source 1..25: mol_type = other DNA
gtcaagatcacagattttgggctgg

SEQ ID NO: 21 - DNA, 62 bp, synthetic construct
  misc_feature 1..62: note = primer
  source 1..62: mol_type = other DNA
atcgaattaattcttaattaattcgaacatcatcaataatataccttattttggattgaagc

SEQ ID NO: 22 - DNA, 62 bp, synthetic construct
  misc_feature 1..62: note = primer
  source 1..62: mol_type = other DNA
caatccaaaataaggtatattattgatgatgttcgaattaattaagaattaattcgatcctg

SEQ ID NO: 23 - DNA, 62 bp, synthetic construct
  misc_feature 1..62: note = primer
  source 1..62: mol_type = other DNA
ccaacggcgttccagacacggttcgaatctcgtaggtcaaggtagtagagtttgcgggcagg

SEQ ID NO: 24 - DNA, 62 bp, synthetic construct
  misc_feature 1..62: note = primer
  source 1..62: mol_type = other DNA
cctgcccgcaaactctactaccttgacctacgagattcgaaccgtgtctggaacgccgttgg

SEQ ID NO: 25 - DNA, 53 bp, synthetic construct
  misc_feature 1..53: note = primer
  source 1..53: mol_type = other DNA
tttactggtaaggctgactgttaaaaaaaaacatggttcttccagctcttccc

SEQ ID NO: 26 - DNA, 53 bp, synthetic construct
  misc_feature 1..53: note = primer
  source 1..53: mol_type = other DNA
cgggaagagctggaagaaccatgtttttttttaacagtcagccttaccagtaa

SEQ ID NO: 27 - DNA, 52 bp, synthetic construct
  misc_feature 1..52: note = primer
  source 1..52: mol_type = other DNA
gaatgtaacactttgacaatgcacaacgcgtggacttctccttcgccgcccg

SEQ ID NO: 28 - DNA, 51 bp, synthetic construct
  misc_feature 1..51: note = primer
  source 1..51: mol_type = other DNA
tgcttaacgggcggcgaaggagaagtccacgcgttgtgcattgtcaaagtg

SEQ ID NO: 29 - DNA, 57 bp, synthetic construct
  misc_feature 1..57: note = primer
  source 1..57: mol_type = other DNA
gctggatcgagatgccgtagagacagcaagacacttgcttgatccaaatccaaacag

SEQ ID NO: 30 - DNA, 57 bp, synthetic construct
  misc_feature 1..57: note = primer
  source 1..57: mol_type = other DNA
tggatttggatcaagcaagtgtcttgctgtctctacggcatctcgatccagcatatc

SEQ ID NO: 31 - DNA, 58 bp, synthetic construct
  misc_feature 1..58: note = primer
  source 1..58: mol_type = other DNA
ccttccttgatgccgaagagggcgacatgtgtcttcacaccctggagcgagtggaccc

SEQ ID NO: 32 - DNA, 59 bp, synthetic construct
  misc_feature 1..59: note = primer
  source 1..59: mol_type = other DNA
tatgagcggataataatttcacacaggaggtttaaactttaaacatgtcaaaagagacg

SEQ ID NO: 33 - DNA, 75 bp, synthetic construct
  misc_feature 1..75: note = oligo
  source 1..75: mol_type = other DNA
ctcatagcgcgtaatactggtaccgctaatagtgactaatagtgactaatagtgatctcacatattattcctttc

SEQ ID NO: 34 - DNA, 94 bp, synthetic construct
  misc_feature 1..94: note = oligo
  source 1..94: mol_type = other DNA
catgtcaagatcacagattttgagggcagaggaagtctgctaacatgcggtgacgtcgaggagaatcctggacctgtgagcaagggcgaggagg

SEQ ID NO: 35 - DNA, 94 bp, synthetic construct
  misc_feature 1..94: note = oligo
  source 1..94: mol_type = other DNA
cctcctcgcccttgctcacaggtccaggattctcctcgacgtcaccgcatgttagcagacttcctctgccctcaaaatctgtgatcttgacatg

SEQ ID NO: 36 - DNA, 50 bp, synthetic construct
  misc_feature 1..50: note = oligo
  source 1..50: mol_type = other DNA
ctcttccgcacccagcagtttggcccgccccttgtacagctcgtccatgc

SEQ ID NO: 37 - DNA, 50 bp, synthetic construct
  misc_feature 1..50: note = oligo
  source 1..50: mol_type = other DNA
gcatggacgagctgtacaaggggcgggccaaactgctgggtgcggaagag

SEQ ID NO: 38 - DNA, 45 bp, synthetic construct
  misc_feature 1..45: note = oligo
  source 1..45: mol_type = other DNA
cacagagggtgctcagaaaggagctttgttccttgggttcttggg

SEQ ID NO: 39 - DNA, 56 bp, synthetic construct
  misc_feature 1..56: note = oligo
  source 1..56: mol_type = other DNA
ggacgaaacaccgtcaagatcacagattttggggttttagagctagaaatagcaag

SEQ ID NO: 40 - DNA, 56 bp, synthetic construct
  misc_feature 1..56: note = oligo
  source 1..56: mol_type = other DNA
cttgctatttctagctctaaaaccccaaaatctgtgatcttgacggtgtttcgtcc

SEQ ID NO: 41 - DNA, 58 bp, synthetic construct
  misc_feature 1..58: note = oligo
  source 1..58: mol_type = other DNA
cgatatctctagactcgaggctaagatacattgatgagtttggacaaaccacaactag

SEQ ID NO: 42 - DNA, 48 bp, synthetic construct
  misc_feature 1..48: note = oligo
  source 1..48: mol_type = other DNA
cgtctgttgtgtgactctggtaactagagatccctcagacccttttag

SEQ ID NO: 43 - DNA, 50 bp, synthetic construct
  misc_feature 1..50: note = oligo
  source 1..50: mol_type = other DNA
cttattttggattgaagccaatatggtttaaacttcgaatctcgtaggtc

SEQ ID NO: 44 - DNA, 50 bp, synthetic construct
  misc_feature 1..50: note = oligo
  source 1..50: mol_type = other DNA
gacctacgagattcgaagtttaaaccatattggcttcaatccaaaataag

SEQ ID NO: 45 - DNA, 40 bp, synthetic construct
  misc_feature 1..40: note = oligo
  source 1..40: mol_type = other DNA
cttacgcatctgtgcggtatttcacaccgcatatggatcc

SEQ ID NO: 46 - DNA, 72 bp, synthetic construct
  misc_feature 1..72: note = oligo
  source 1..72: mol_type = other DNA
ctgaataattttgtgttactcatagcgcgtaatatttgtctagggccgcggggactttgaccgtttacgtgg

SEQ ID NO: 47 - DNA, 46 bp, synthetic construct
  misc_feature 1..46: note = oligo
  source 1..46: mol_type = other DNA
cgccccaggccataagctaatagtgactaatagtgactaatagtga

SEQ ID NO: 48 - DNA, 44 bp, synthetic construct
  misc_feature 1..44: note = oligo
  source 1..44: mol_type = other DNA
cactattagtcactattagtcactattagcttatggcctggggc

SEQ ID NO: 49 - DNA, 45 bp, synthetic construct
  misc_feature 1..45: note = oligo
  source 1..45: mol_type = other DNA
gacgtcgaggagaatcctggacctgaaaccagagggcgaagaccg

SEQ ID NO: 50 - DNA, 45 bp, synthetic construct
  misc_feature 1..45: note = oligo
  source 1..45: mol_type = other DNA
cggtcttcgccctctggtttcaggtccaggattctcctcgacgtc

SEQ ID NO: 51 - DNA, 50 bp, synthetic construct
  misc_feature 1..50: note = oligo
  source 1..50: mol_type = other DNA
cgcgcaaaacccctaaataagggcgggccaaactgctgggtgcggaagag

SEQ ID NO: 52 - DNA, 50 bp, synthetic construct
  misc_feature 1..50: note = oligo
  source 1..50: mol_type = other DNA
ctcttccgcacccagcagtttggcccgcccttatttaggggttttgcgcg

SEQ ID NO: 53 - DNA, 39 bp, synthetic construct
  misc_feature 1..39: note = oligo
  source 1..39: mol_type = other DNA
ggggcctttcctgttccttaggtgtccttgacagcagtg

SEQ ID NO: 54 - DNA, 75 bp, synthetic construct; identical to SEQ ID NO: 33
  misc_feature 1..75: note = primer
  source 1..75: mol_type = other DNA

SEQ ID NO: 55 - DNA, 38 bp, synthetic construct
  misc_feature 1..38: note = primer
  source 1..38: mol_type = other DNA
gaggagaatcctggacctaagttcctggtgaacgtggc

SEQ ID NO: 56 - DNA, 38 bp, synthetic construct
  misc_feature 1..38: note = primer
  source 1..38: mol_type = other DNA
gccacgttcaccaggaacttaggtccaggattctcctc

SEQ ID NO: 57 - DNA, 38 bp, synthetic construct
  misc_feature 1..38: note = primer
  source 1..38: mol_type = other DNA
caagaggaagaggcagcagggcgggcgggccaaactgc

SEQ ID NO: 58 - DNA, 38 bp, synthetic construct
  misc_feature 1..38: note = primer
  source 1..38: mol_type = other DNA
gcagtttggcccgcccgccctgctgcctcttcctcttg

SEQ ID NO: 59 - DNA, 45 bp, synthetic construct
  misc_feature 1..45: note = primer
  source 1..45: mol_type = other DNA
cccaagaacccaaggaacaaagctcctttctgagcaccctctgtg